(19) United States
(12) Patent Application Publication
(10) Pub. No.: US 2020/0338263 A1
(43) Pub. Date: Oct. 29, 2020

(54) Automated Medical Infusion Device and Method with Improved Accuracy and Safety Characteristics and MRI-Safe Capability

(71) Applicant: Michael H. Wollowitz, Chatham, NY (US)

(72) Inventor: Michael H. Wollowitz, Chatham, NY (US)

(73) Assignee: Michael H. Wollowitz, Chatham, NY (US)

(21) Appl. No.: 15/843,989

(22) Filed: Dec. 15, 2017

(51) Int. Cl. A61M 5/168; A61M 5/14; A61M 39/28; A61M 39/22

(52) U.S. Cl. CPC A61M 5/16813; A61M 5/1411; A61M 5/16831; A61M 5/1689; A61M 39/22; A61M 2205/502; A61M 2205/52; A61M 39/28

(57) ABSTRACT

A medical infusion device and related method of use for controlling the flow rate of a fluid into a patient's body, comprising: a tube for carrying fluid from a proximal end to a distal end thereof under the action of a driving pressure, which tube is flexible or has a flexible segment at some point along its length; a clamping element capable of preventing fluid flow by fully occluding a portion of the flexible tube or the flexible segment; a movable pusher element for acting variably against the clamping element to variably reduce the occlusion of the clamped portion of the flexible tube or flexible segment and thereby provide a controlled rate of fluid flow; two independently controllable electromechanically controlled actuator elements capable of moving variably over a prescribed range; and a mechanical linkage among the actuator elements, the pusher element and the clamping element.

### CROSS REFERENCE TO RELATED APPLICATIONS

This application claims priority benefit of pending provisional application U.S. 62/435,991 filed Dec. 19, 2016, and also of pending provisional application U.S. 62/558,266 filed Sep. 13, 2017. These priority applications are hereby incorporated by reference in their entireties.

### BACKGROUND OF THE INVENTION

The present invention is in the field of medical drug and fluid infusion, in particular the delivery to a patient, typically by intravenous infusion, of fluid from bags or bottles. Further, the field of this invention includes, but is not limited to, delivery of fluid to a patient, typically by intravenous infusion, in environments where use of ferrous and magnetic materials must be minimized.

Intravenous delivery of drugs and other fluids to patients is a common and important medical practice. For delivery of larger fluid quantities, typically over 100 mL, these fluids are typically delivered from a container in the form of a sterile bag or bottle. Usually the bag or bottle is hung from a pole or rack at a height somewhat above the seated or prone patient. Connection between the container and patient is made with an IV set. This is most basically a long, flexible polymer tube with a spike fitting at one end and a Luer fitting at the other. The spike is used to make a sealed, sterile connection to the fluid container; a hypodermic needle is mounted to the Luer fitting to deliver the fluid to the patient's venous system. The IV set may also incorporate various other elements including clamps, valves, filters, additional ports.

It is important for both safety and efficacy to provide control of the flow of fluid from the container to the patient. The flow requirement is typically specified as a flow rate with units of milliliters per minute or milliliters per hour, but may also be specified as a total delivery time and volume. A variety of methods have been developed to provide accurate and reliable control of the flow.

The oldest and most basic method uses gravity to drive the flow. With the container located above the patient there is a static pressure ΔP=ρgh, where ρ is the density of the fluid, g is the acceleration of gravity, and h is the height of the container relative to the patient. Due to this pressure fluid will move from the container to the patient at a rate determined by the geometry of the IV set. For typical use a drip chamber is used to measure the flow rate. The drip chamber is typically positioned just below the spike on the IV set, but may also be placed at an intermediate position along the tube of the IV set. The drip chamber is a clear plastic tube, usually 1-2 centimeter diameter and 5-10 centimeter length, closed at both ends and, for use, oriented vertically. Fluid enters at the top of the drip chamber and passes through a small nozzle. It falls from the nozzle as individual drops. The drops land in a small pool of fluid at the bottom of the drip chamber and the fluid then passes into the connecting tube to the patient. The nozzle is constructed such each drop will be of a known volume, typically 0.1, 0.05, or 0.017 mL. A clinician can look through the clear wall of the drip chamber and view the drops as they form and fall. A close estimate of the flow rate can be made by using the known volume per drop and by counting either the time between drops or the number of drops in a fixed time interval.

To control the flow a manually-operated, adjustable clamp is placed on the tube at a point below the drip chamber. The clamp acts to flatten and partially occlude a short portion of the tube, increasing the resistance to fluid flow. The clinician adjusts this clamp and notes the effect on the drop rate. With some skill the clinician can make a series of adjustments to produce a desired drop rate and so, in turn, a desired flow rate or delivery time.

Manually-controlled gravity-driven infusion, using a drip chamber for measurement and an adjustable clamp for control, is simple and inexpensive. It has been in widespread use since the 1950's and continues to be used for some non-critical applications and where low cost is an overriding factor. In other cases it has been superseded because of several critical limitations:

Setting the flow rate is time-consuming and not very accurate. The setting requires repeated trial-and-error adjustments while manually counting drops.

After the initial setting there is no ongoing measurement or control so the flow rate may vary over time as, for example, the fluid level drops in the container, the fluid temperature changes, or the flexible tube re-conforms in the area under the clamp. The last item is most critical, the polymer tube will re-conform over time to relieve applied stress; this can lead to either an increase or decrease in flow rate.

Being completely manual this method cannot generate a signal or alarm when a dose has been completed or if there is a problem such as a blockage that prevents flow. Unsafe conditions cannot be detected and the flow cannot automatically be stopped if there is an unsafe condition or if a desired total dose has been delivered.

There is no automation to verify dosage or flow rates. All steps and calculations are performed manually, so there is a high likelihood of human error.

Since the 1970's infusion pumps have replaced manually-controlled, gravity-driven infusion for many applications. The pumps are of various types, but share the important characteristic that they have a pumping chamber built in to the disposable IV set. This is necessary to maintain sterility of the fluid path. The pump mechanism works externally to this sterile pumping chamber to pump a nominally constant volume of fluid during each pumping cycle. The volume pumped per cycle varies from about 0.1 mL to 2 mL per cycle. Most pumps are either of the peristaltic type, which use rollers or moving fingers to push fluid along a length of flexible tubing, or diaphragm type, which have a flexible diaphragm or bulb that is pressed to move fluid and a set of valves to control the direction of fluid movement. Modern infusion pumps are computer controlled and have a sophisticated user interfaces and programming. They are designed to assist with rate and dosage setting, prevent certain user errors, and detect and respond to certain unsafe conditions during operation.

Infusion pumps also have several limitations, which are different than those encountered with gravity-driven infusion:

Pumps can generate excessive pressure. Unlike gravity-driven infusion in which the maximum pressure is fixed by the height of the fluid container, pumps can generate high pressures, particularly if there is a partial or complete blockage in the line or at the IV needle. This can present a danger to the patient. At typical low operating pressure the pumps may perform more work flexing a tube or diaphragm than actually pumping fluid, so they are relatively insensitive to changes in fluid pressure. Additional sensors may be added to monitor fluid pressure but they add cost and complexity and are not always reliable.

Pumps can inadvertently pump air as well as fluid, resulting in a risk of embolism. If there is air in the container or a leak in a connection, a constant volume type of pump can cause a significant amount of air to enter the patient's bloodstream, which can result in heart failure or stroke. Sensors may be added to the pump mechanism to detect air in the IV set, but this adds cost and complexity and an additional potential failure point. It may be noted that with gravity-drive infusion the flow will slow and then stop as the fluid level in the container and IV set decreases; there is no danger of air being forced into the patient's blood stream.

Most medical pumps calculate rather than measure fluid flow. Flow is calculated based on the number or rate of pump cycles and the expected volume per pump cycle. In case of an empty container, leak, or blockage the actual flow may be very different than the calculated flow. Also, most pumps are only nominally of constant volume type; variations in container height, backpressure, or fluid viscosity can produce significant errors in actual flow rate versus calculated flow rate.

Pumping mechanisms are complex and require precision manufacturing and calibration in order to provide reasonable accuracy. The pumps are also generally inefficient, in terms of the energy used relative to the work done on the fluid. Most large volume pumps can only operate for short periods of time on batteries and must be connected to an external power source during normal operation.

An additional limitation is that most medical pumps use magnetic motors, typically of the stepper motor or brushless servo motor types, to drive the pump mechanisms. The ferrous and magnetic materials in these motors present problems when they are used in high magnetic field environments, particularly in Mill facilities. The amount of ferrous and magnetic material present creates a safety hazard and may also degrade the imaging ability of the MM.

An infusion pump developed by Iradimed uses a non-magnetic motor of an ultrasonic, piezoelectric type, and is specifically intended for use in MM environments. This is an effective solution but a motor of this type with sufficient output power to drive the pump mechanism is relatively large and expensive compared to magnetic motors of similar power. The motor requires a high voltage signal to provide sufficient torque; this high voltage signal is generated by magnetic components located at a distance from the MM system and connected to the pump by long cables. Also, piezoelectric motors operate with a rubbing action between fixed piezo elements and a moving rotor. The rubbing action causes wear and reduces the operating life of the motor compared to stepper or brushless magnetic motors.

While infusion pumps have become the general standard for medical infusion, significant work has also been done to add automated control to gravity-driven infusion. Development has occurred in three areas: automated measurement of flow, automated control of flow, and incorporation of the type of “user friendly” interfaces and error-prevention functions as used in modern infusion pumps.

Automated gravity-driven infusion has several advantages:

Incorporation of an automated drop counter or other automated flow measurement provides continuous monitoring of actual flow status. Flow rates are measured rather than being calculated, with the potential for improved accuracy. Conditions such as leaks, blockages, or empty fluid containers can be quickly detected by a change in the actual flow rate.

Flow stops when there is not more fluid to deliver. There is no risk, as with a pump, of delivering air to the patient's venous system. A sensor may be included to detect air in the IV set, but it is not critical for patient safety.

Power consumption may be greatly reduced compared to infusion pumps. A motorized clamp or pinch valve is opened once at the beginning of the infusion and then, typically, only adjusted occasionally to maintain or change the flow rate. The clamp motor runs for a small fraction of the time of a continuously operating pump motor and, further, it can typically operate at lower power. This makes it practical to operate the gravity driven system for extended periods on batter power, improving mobility and ease of use.

The mechanism is much simpler than that of a typical pump and requires little calibration. Because there is continuous feedback from an automated drop counter or other automated flow measurement the control is of a closed-loop type and can adjust as needed to maintain a desired flow rate. A nominally constant volume pump is an open-loop system; because it has no feedback its accuracy must be “built in” by precision fabrication and careful mechanical or electronic calibration.

Previous automated gravity-drive infusion has also had several limitations that are addressed in several novel ways by the present invention to be disclosed here:

In case of a mechanical, electrical, or software failure, a gravity-drive infusion device may continue to deliver fluid without ongoing flow control; an open valve or clamp may remain open but uncontrolled. In contrast, infusion pumps are normally configured so that, once the IV set is mounted to the pump, flow cannot occur when the pump is not operating or fails to operate. It is normally expected that an automated infusion device will respond to a failure condition by stopping all fluid flow and, if possible, raising an alarm signal. The present invention provides a method of redundant mechanical and electronic components that monitors for error or failure conditions and responds appropriately. Typically flow is stopped, but other responses can be made available.

Similarly, there is a risk if an automated gravity-drive infusion device shuts down with the control mechanism in an incorrect state. When a new IV set and fluid container are loaded, that fluid flow will begin immediately rather than when it is programmed to begin. This may deliver an undesired and possibly dangerous fluid volume to a patient. The present invention provides a robust interlock system that prevents flow from occurring under such conditions.

It is important for safe and correct operation of an infusion device that the IV set always be correctly positioned and aligned when mounted to the device to provide correct pumping or clamping actions. This has been highly developed in infusion pumps but had been only partially developed for automated gravity-driven infusers. The present invention provides means for simple and positive attachment of the flow control and flow monitoring parts of the IV set to the control mechanism.

Previous automated gravity-driven infusion devices have contained magnetic motors and other ferromagnetic components, making them incompatible with use in high magnetic field environments such exist in MM facilities. The present invention provides means for operation with non-magnetic motors and reduction or elimination of other ferromagnetic components.

### SUMMARY OF THE INVENTION

Disclosed herein is a medical infusion device and related method of use for controlling the flow rate of a fluid into a patient's body, comprising: a tube for carrying fluid from a proximal end to a distal end thereof under the action of a driving pressure, which tube is flexible or has a flexible segment at some point along its length; a clamping element capable of preventing fluid flow by fully occluding a portion of the flexible tube or the flexible segment; a movable pusher element for acting variably against the clamping element to variably reduce the occlusion of the clamped portion of the flexible tube or flexible segment and thereby provide a controlled rate of fluid flow; two independently controllable electromechanically controlled actuator elements capable of moving variably over a prescribed range; and a mechanical linkage among the actuator elements, the pusher element and the clamping element, configured such that the motion of the actuator elements is transmitted to the pusher element which in turn is transmitted to the clamping element for varying the occlusion and thereby varying the rate of fluid flow; wherein: the motion of the pusher element is a function of the motions of the actuator elements; and the force applied by the pusher element is a function of the force applied by the actuator elements.

Also disclosed are specific variants of the invention using a non-magnetic electrical motor for use in environments where ferromagnetic material and extraneous magnetic fields must be minimized, and using separable modules.

### BRIEF DESCRIPTION OF THE DRAWINGS

The features of the invention believed to be novel are set forth in the appended claims. The invention, however, together with further objects and advantages thereof, may best be understood by reference to the following description taken in conjunction with the accompanying drawing(s) summarized below.

FIG. 1 shows an implementation of the overall device with the cassette assembly in place.

FIG. 2 shows an implementation of the overall device without the cassette assembly.

FIG. 3 shows the internal structure of the overall device.

FIGS. 4A-4C show the cassette assembly in full, exploded, and cut-away views.

FIGS. 5A-5D show the flow control mechanism in several orientations.

FIG. 6 is a simplified view of the flow control mechanism and cassette assembly, showing the interaction of the components.

FIGS. 7A and 7B show detail of the cam component of the flow control mechanism.

FIG. 7C shows an alternative “V-profile” form of the cam.

FIG. 8 is a simplified diagram of the electronic and electrical components of the device, showing power and signal connections.

FIGS. 9A-9D show a means for mounting the cassette assembly including detail of the mounting features of the mounting component and cassette assembly, individually and combined.

FIGS. 10A-10D show a sequence of steps for mounting the cassette assembly, which may be reversed for unmounting.

FIGS. 11A-11C show the mechanical interlock means that prevents mounting of the cassette assembly when the device is in an incorrect state.

FIG. 12 shows an implementation of a weight sensor for use with the device, with its enclosure partially removed to reveal internal detail.

FIG. 13 shows and alternative implementation of the control mechanism and cassette assembly where the action of the cams is reversed and where no spring is required in the cassette assembly.

FIGS. 14A-14B show an alternative implementation employing digital imaging to detect drops, fluid levels, and other information from the drip chamber of the cassette assembly.

FIGS. 15A-15C show simplified views of specular and diffuse reflections from a pendant drop as recorded by a digital imager, as the pendant drop grows and then breaks away from the nozzle in a drip chamber.

FIG. 15D shows a simplified view of specular and diffuse reflections from fluid exiting the nozzle of the nozzle in a drip chamber under a “free flow” condition.

FIG. 16A is a plot of an idealized displacement signal of specular highlights at a low drop rate.

FIG. 16B is a plot of an idealized displacement signal of specular highlights at a higher drop rate with a transition to a free-flow condition.

FIG. 17 is a flow chart showing the use of a digital imaging implementation to provide feedback for flow rate control and detection of error conditions.

### DETAILED DESCRIPTION

General Overview:

Medical delivery of drugs and other fluids from bags and bottles is typically performed using either automated pumps or manually controlled gravity-driven apparatus. The present invention employs gravity-driven flow but has features similar to automated medical pumps. It provides similar or better flow accuracy and potentially improved safety features relative to existing medical infusion pumps. It is referred to in this document as the “IV Controller” because it specifically controls fluid flow rather than generating flow as a pump would do.

The basic principal of the IV Controller is that fluid flow is monitored by one or more sensors and that gravity driven flow is regulated using an automatically adjustable valve. A computerized control system uses feedback from the sensor or sensors to modify the valve restriction to maintain or adjust the flow rate to a desired value. Central to the invention is a mechanism for adjusting the valve restriction that is simple, accurate, and robust. It provides specific safety features. It also requires small and low power motors, relative to those in typical infusion pumps, and so is advantageous for operation without main power or for operation in environments such as MRI facilities where use of ferrous and magnetic materials must be minimized.

The flow sensing means are a drop counter and optionally a weight sensor. The drop counter uses a drip chamber of known type and uses optical sensing to detect each individual droplet falling in the drip chamber. This provides rapid feedback of flow rate but may have a long term error due to variations in drop size. The weight sensor has a hook or loop on which an IV bag or bottle is hung; the weight sensor measures the weight of this container and its contents and so monitors fluid flow as the change in weight over time. The weight sensor typically has insufficient resolution to provide rapid feedback (relative to the drop counter) but it provides precise longer-term measurements. Using the two sensors together provides both rapid feedback and long term precision as well as providing redundancy in case of a malfunction of either sensor. The orthogonality of the two sensing means further improves safety because each sensing means is generally immune to factors that may cause malfunction of the other sensing means. For example, a very high rate of flow may cause within the drip chamber a “free flow” condition, a continuous stream of fluid without individually-detectable or visible drops. The drop counter may be unable to measure this free flow but the weight sensor will detect a change in container weight. Alternative embodiments of this invention may use only one of the two sensors, depending on requirements for accuracy, redundancy, and ease of use.

The drop counter is typically of an optical type, consisting of a light source, typically an infrared LED, and a light detector, typically a photo-transistor with a filter to block visible wavelength light. The source and detector are arranged either in transmission mode, where a falling drop momentarily causes a reduction of source illumination reaching the detector, or a reflective mode, where a falling drop momentarily reflects source illumination toward the detector. In either case, the resulting electrical signal is processed and drop is counted when the signal matches some combination of defined parameters. Multiple light sources, light detectors, or other related components may be employed to improve reliability of drop detection and to avoid false drop detections.

Any alternative type of drop counter that can reliably detect individual drops may also be employed. One alternative is to employ one of more digital imaging devices, or digital cameras, with appropriate light sources. A camera may be directed toward drop forming portion of the drip chamber to record the formation and breaking away of droplets. Alternately or additionally a camera may be directed toward the surface of the fluid pool at the bottom of the drip chamber to detect disturbances as drops strike the surface as well as the level of the fluid surface. This alternative is discussed in greater detail below.

Physical Description:

The IV Controller generally consists of three main physical elements: a main controller, a cassette that is part of a IV delivery set, and optionally a weight sensor. The cassette contains the flow control valve. The main controller consists of sub elements including a computerized electronic controller, a valve control mechanism, components to mount the cassette, and several sensors. The computerized electronic controller provides means for programming various infusion parameters, such as flow rate, volume, and time, and automatically controlling the infusion process to maintain these parameters while also monitoring for potential faults or errors. A user interface may be included in the main controller or may be located remotely. The weight sensor is typically separate from the main controller but may receive electrical power from it. The weight sensor communicates with the main controller using wired or wireless (radio or optical) means.

The main controller is typically mounted to e.g. an IV pole at a convenient height for user operation. The weight sensor provides a hanging point for the IV container and so is typically mounted at a higher level, similar to standard gravity feed IV configurations. The higher level may be preferred to provide adequate head height between the IV fluid source and the entry point to the patient to achieve required flow rates. The IV set tubing between the proximal bag spike and the central cassette must of sufficient length that there will be some slack along its length when in use; this ensures that there will be not tension on the IV container that could affect the weight measurements.

A critical part of the invention is the combination of the flow control valve and the associated control mechanism. These are described in detail below but an overview is given here. The valve is within the removable cassette, below the drip chamber, and in the fluid path between the drip chamber and the distal portion of the IV set. It consists of a short length of flexible tubing that fits between a fixed stop and a movable valve blade. The valve blade is pressed against the stop by a compressed spring, pinching the tube so that it is fully occluded and so blocking any fluid flow. A portion of the valve blade is exposed outside of the cassette. This portion can be pushed in, opposing the spring force, and allowing the tube to open to allow fluid flow. The valve blade may be moved in small increments causing corresponding small changes in fluid resistance and so making fine adjustments or corrections to the flow rate.

The cassette is mounted to the main controller using features that are described in detail below. The cassette is mounted, delivery parameters programmed by the user, and operation initiated. At this time the control mechanism is activated.

The control mechanism is contained within the main controller with the end of one element, the pusher, exposed and directed at the exposed portion of the valve blade. The mechanism acts to extend the pusher to push in the valve blade to initiate and control fluid flow. The total movement of the pusher along its axis of travel from fully retracted (no flow) to fully extended (maximum flow) is approximately 2.5 mm with most flow adjustment occurring in a sub-range of less than 0.5 mm. To allow accurate control over a wide flow range the resolution of the pusher movement must be small—on the order of 0.1 mm or less. While resolution must be high, feedback from the flow sensors allows correction for errors in positional accuracy, making this parameter less critical.

In one embodiment, the mechanism employs two specially shaped cams, mounted on the same plane and on opposite sides of the pusher and equidistant from it. Each of the cams has a fixed, well supported rotational axis and is rotated by a motor, typically coupled to the cam by a gear reducer. The gear reducer both increases torque from a typically small, low-power motor and increases the rotational resolution of the cam. The cams act against a cross arm, which is a beam that is generally perpendicular to the pusher and is connected to it by a pivot so that the two components move together but the cross arm can pivot over a small angle relative to the pusher. An arrangement with an overload spring allows the pusher to move relative to the cross arm in case of excessive loads against the pusher. A rotating cam follower may be mounted at each end of the pusher arm to minimize friction where the two cams act against the cross arm. A second, light spring may be attached to either the pusher arm or pusher rod to keep the cam followers lightly seated against the cams.

As one or both cams rotate to advance to move the pusher arm to the right the drive rod is thus also driven to the right, as long as no force is encountered that exceeds the pre-load on the overload spring. The rightmost end of the drive rod is shaped to act to press in the valve blade of the cassette. A stop feature on the drive rod limits the motion of the drive rod such that it can never press in the valve blade more than a designed distance.

In normal operation the cassette is inserted with the pusher rod fully retracted and not touching the valve blade. At the beginning of operation the pusher rod is advanced by both cams moving together until it reaches a working position, typically near the point at which flow is detected. For the remainder of the infusion one cam remains fixed, acting as a fulcrum for the pusher arm. The other cam then rotates, causing a small tilt in the pusher arm and moving the pusher rod about one-half of the distance of any corresponding rise in the cam. The pusher arm has clearance around the pusher rod such that the pusher rod will not have interference with the pusher rod or overload spring even at the maximum tilt where one cam is at the fully retracted position and the other at the fully extended position.

The pre-load on the overload spring must be somewhat greater than the maximum force that will be exerted on the valve blade during normal operation. The spring will only be further compressed, allowing the drive rod to slide relative to the drive bar, in one of several potential overload conditions.

One aspect of this configuration is that if an error or failure is detected in the drive mechanics or electronics for either cam then the other cam can immediately be rotated to the fully retracted position. The dimensions of the mechanical components are chosen so that when either cam is in the fully retracted position the drive rod will not contact the valve blade no matter what the position of the other cam, so flow can always be prevented. The drive electronics for the cams must be mutually independent and also independent of the main controller for maximum redundancy in case of error or failure.

Another aspect of this configuration is that if both cams attempt to move to the fully advanced position at the same time, a stop feature will limit the travel of the pusher so that the cassette is not damaged, mechanically trapped, or otherwise abused. In this case either the cross arm will advance and the spring be compressed or one or both of the motors will stall, with the pusher moving only a limited distance in either case. This protects against a “worst case” condition involving simultaneous failure of multiple sub-systems and ensures that the user will be able to remove stop fluid flow by removing the cassette from the controller.

Another aspect of this configuration is that if the pusher is extended (e.g. from an incorrect shut down of the system) and the user attempts to force it manually to the retracted position (by pressing hard on the exposed end of the pusher) the pusher can momentarily move by compressing the overload spring. When the manual force is released it will return to the extended position. The compression of the spring will prevent excessive forces from being transmitted to the cams, cam followers, and drive components where it might otherwise cause damage.

It will be noted that the mechanism described here is of a generally known additive type in which three linear or rotating elements are connected such that motion of one element is proportional to the sum of the motions of the other two elements. This type of mechanism can be realized in many ways using gears, lead screws, cams, wedges, rollers or other mechanical elements. The present invention includes any mechanical means or linkage with the following two characteristics:

First, the action of two independent motion inputs are combined into motion of a single output for the purpose of variably operating a valve to control flow of a medical fluid, such that the combination takes the form:

Xₒᵤₜ=k₁Xᵢₙ₁+k₂Xᵢₙ₂+E(Xᵢₙ₁,Xᵢₙ₂)

where Xᵢₙ₁ and Xᵢₙ₂ are the input displacements, Xₒᵤₜ is the output displacement, k₁ and k₂ are multiplicative factors related to mechanical features such as gearing or lever-arm length, and E is a function that accounts for minor variations due to non-linear motions (e.g. tilting of the pusher arm).

Second, the geometry of the mechanical elements is such that both inputs are required to initiate flow but that either input can act independently to stop or adjust fluid flow.

It may further be noted, however, that the embodiment of the invention described in detail here is advantageous in that it is simple in design and construction and minimizes the need for precision fabrication and calibration. In particular, the more precise and complex components, such as the gear reducers, are duplicated and independent of each other, providing redundancy, while the common elements that create the combined output are very simple and robust, providing a negligible risk of failure.

While the present invention is generally described as a medical infusion device the invention may also be applied to other medical fluid delivery systems or to non-medical fluid control systems that require precise flow control and high reliability.

Safety Considerations:

Safety of an automated medical device is typically seen as depending on having means to detect and respond to all likely malfunctions and having redundancy to minimize risks of single-point failures. Equally critical, however, is the need for robust design of the device. Robust design typically includes the following aspects:

Reduction of complexity (particularly mechanical complexity) wherever possible.

Ruggedness of construction and minimization of internal forces and force paths.

Minimized requirements for critically adjusted or calibrated components.

Minimized number of potential failure points.

The principles of robust design may conflict with notions of installing additional sensors or redundant mechanisms. Additional components may protect from errors but they may also create errors. A balance must be achieved. Good application of robust design principles may avoid potential conflict by intrinsically reducing requirements for sensor and redundant mechanisms.

The invention applies robust design principles in this manner. The mechanical systems are simple and protected from damage. Forces are relatively low and mainly act along a single plane. Components can be made rugged. The dual motor and dual cam positioning system provides safe handling of possible malfunctions and maintains simplicity by duplicating components rather than employing geometrically-different components. The mechanical interlock system prevents incorrect cassette insertion with a minimum of additional components or potential failure points. The cassette itself is simple and its mounting system requires few components and few moving parts. If properly constructed, the mechanisms require little or no adjustment or calibration before use and so cannot lose calibration during use.

The use of gravity-driven flow with two different flow sensors provides other levels of safety. In this case there is both redundancy and orthogonality to protect against flow sensor malfunction. Certain failures that may require additional protection on infusion pumps are not critical here. For example, if a tube clamp is left in place or set in a pump then the device can operate for some time before the lack of flow is detected. With the present invention the flow sensors will quickly detect the error. Infusion pumps typically require air-in-line sensors to avoid pumping air when all fluid has been expelled. With a gravity-driven infuser the flow will stop immediately when the weight of the fluid has been eliminated. An air-in-line sensor is therefore not required in the present invention, although one could be added to fit specific user or certification requirements.

### Primary Embodiment

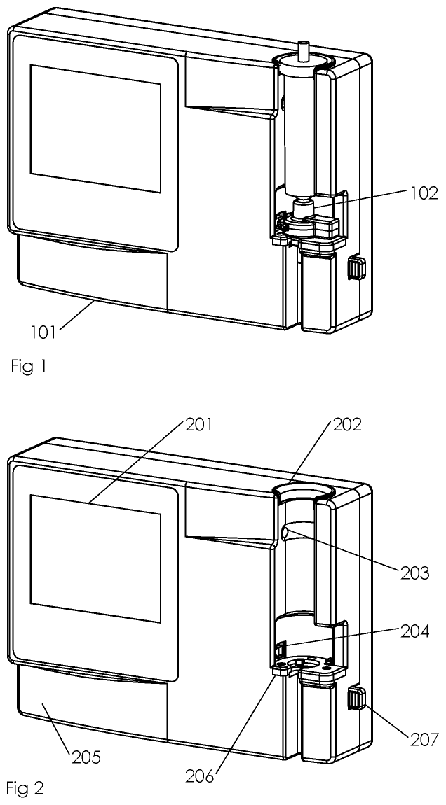

Major components: FIG. 1 shows an overall embodiment of the invention, consisting of IV controller 101 and cassette assembly 102. The cassette assembly 102 is normally part of a disposable IV set, which includes flexible tubes connected to the upper and lower ends of said cassette assembly. The tube connecting to the upper end of the cassette has, at its proximal end a spike for connection to a standard type of IV bag or bottle. The tube connecting to the lower end may terminate in a standard Luer-lock fitting and may have additional ports, clamps, or filters, all of standard type. The general configuration of the tubing and other IV set components are well known and are not shown in these illustrations.

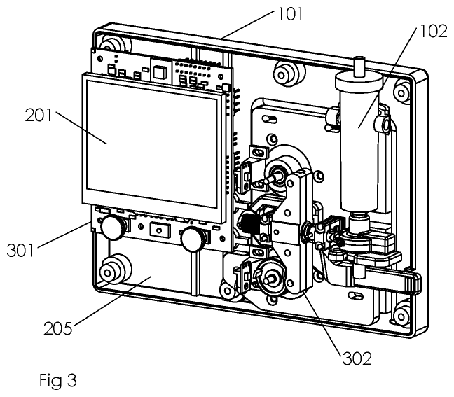

FIG. 2 shows the major features visible from the outside front surface of IV Controller 101. These includes user additional control 201, which may be a touch-screen type of user interface. Cassette mounting component 206 is employed to mount and constrain the lower portion of cassette 102 and locating feature 202 is employed to constrain the upper end of the cassette. Optical drop detector 203 is employed to detect drops forming and falling within the drip chamber component of the cassette. The actuating tip of the “pusher” component 204 protrudes from the interior of the IV controller. Optional manual flow control lever 207 allows manual override of the controller. All these features are described in greater detail below.

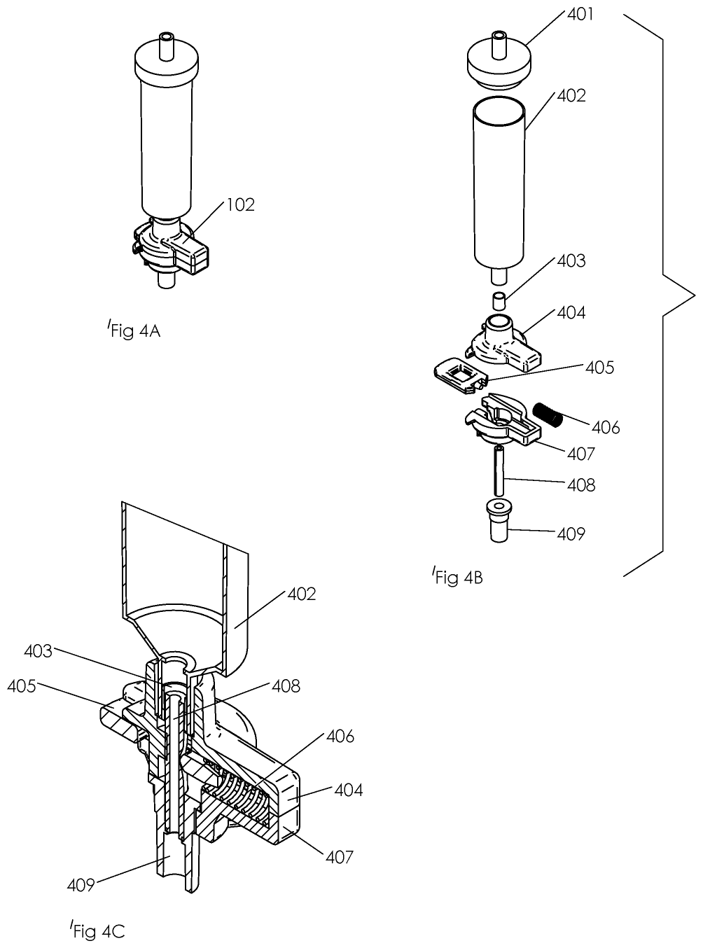

FIG. 3 shows the interior of IV controller 101 with cassette assembly 102 also shown. Visible within the IV controller are control and display electronics 301, which consists of a flat-panel display, user input elements such as touch-screen input and push buttons, a micro-controller of known type, and electronic interfaces of known type for interaction with sensors, motors, and other electronic or electromechanical devices. Also visible is control mechanism 302, described in detail below. Control mechanism 302 and cassette mounting component 206 are fixed within enclosure 205 and are aligned to each other, as will be further described.

Cassette Assembly:

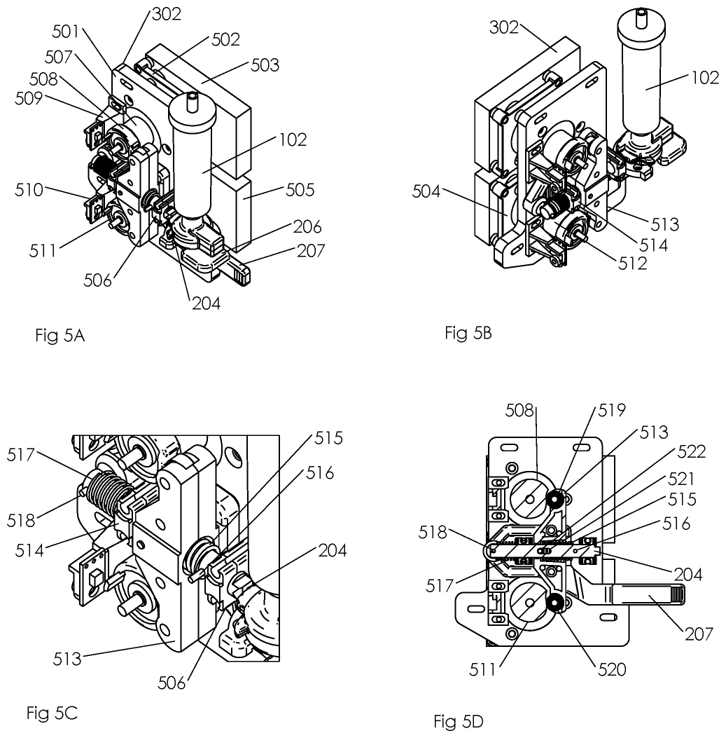

FIG. 4a shows the complete cassette assembly 102. FIG. 4b shows the components of the cassette assembly in an exploded view. Uppermost are drip chamber cap 410 and drip chamber body 402, which may be of a standard type, e.g. DirectMed Corporation model DC-008 cap and model DC-021a body, or modified for this use. Upper cassette body 404 and lower cassette body 407 are joined together and contain upper tube adapter 403, valve blade 405, compression spring 406, flexible tube 408 and lower tube adapter 409. Upper cassette body 404 joins to the outer surface of the small cylindrical section at the bottom of drip chamber body 402.

Cassette assembly 102 is incorporated into an IV set of generally standard type. These additional components of the IV set are of variable type, depending on the specific intended medical use and are not shown in these figures. Generally an additional flexible tube is joined to the top of drip chamber cap 401; a standard bag or bottle attachment spike is joined to the opposite end of this tube, forming the proximal end of the IV set. A further additional flexible tube is joined to the bottom of lower tube adapter 409; additional fittings of standard type are joined to this tube to form the distal end of the IV set which may include valves, clamps, filters, and additional ports and which generally terminates in a Luer fitting to which an IV needle may be attached.

FIG. 4c shows the detail and operation of the flow control elements of cassette assembly 102. Valve blade 405 fits around flexible tube 408 and slides along guides within the joined upper cassette body 404 and lower cassette body 407. Compression spring 406 is contained in a spring retainer section of the joined bodies where it is held a compressed state against the rear of the valve blade. Flexible tube 408 is pressed by the between the valve blade and a valve stop that is part of the body sections, pressing the tube flat at this point so that it is fully occluded to prevent potential fluid flow. Because of the small diameter, flexibility of the tube, and relatively low pressure differential, which is due only to head height, a spring force of about 1-4 pounds is sufficient to ensure complete occlusion. The front end of the valve blade projects from the body; it may be pressed inward, moving the valve blade inward, compressing the valve spring, and allowing the flexible tube to become controllably non-occluded to allow fluid flow.

Flexible tube 408 is made from highly flexible elastomer with low “memory,” such as a standard type of low-durometer silicone tubing. It is joined to upper tube adapter 403 and lower tube adapter 409 using adhesive or mechanical seals. The flexible tube is typically about 2 cm in length and 3 mm to 5 mm outside diameter. The upper tube adapter is joined to the inner surface of the small cylindrical section at the bottom of drip chamber body 402. Alternatively, the small cylindrical section may be modified or sized to fit directly to the flexible tube, eliminating the need for the upper tube adapter.

Control Mechanism:

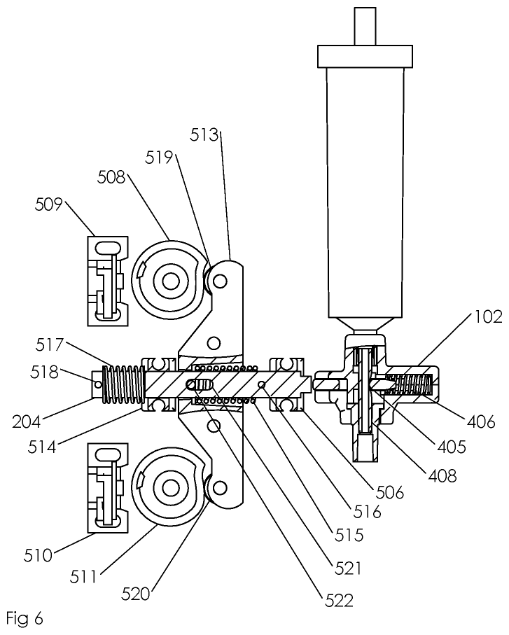

FIGS. 5A through 5D show detail of control mechanism 302. These several views are shown to display all the components of this mechanism. FIG. 5A is a top-left view, FIG. 5B is top-right view, FIG. 5C is a detail view of FIG. 5A, and FIG. 5D is a plan view in partial cross section. Cassette assembly 102 and cassette mounting component 206 are also shown in views 5A through 5C to show the relationship between the components. The overall structure of control mechanism 302 is as follows:

Mounting plate 501 is fixed within enclosure 205. Components are fixed to mounting plate 501 to form an upper actuator assembly. Upper motor 503 is fixed to upper motor adapter 504, which is in turn fixed to mounting plate 501 and contains upper gear reducer and bearing 507. Upper cam 508 is mounted on the output shaft of upper gear reducer 507. Upper optical detector 509 is fixed to mounting plate 501 adjacent to upper cam 508 and is used to detect a reference or home position of upper cam 508.

A lower actuator assembly is similar or identical in form to the upper actuator assembly and is similarly disposed. It consists of lower motor 505, lower motor adapter 504, lower gear reducer and bearing 512, lower cam 511, and lower optical detector 510.

Motors 503, 505 are, in this embodiment, non-magnetic motors of known piezoelectric or ultrasonic type. Exemplar motors are of the type manufactured by PCBMotor ApS, Ballerup, Denmark. These exemplar motors contain no magnetic material and minimal ferrous material and have built in micro-controllers and optical encoders. Gear reducers and bearings 507, 512 may employ gearing of various types including parallel shaft, right angle, worm gear, or planetary gear and may be of simple of compound type. A reduction ratio of about 5:1 to 12:1 has been determined to perform well with the exemplar motors but may be changed for use with other motors. The bearings are chosen to provide support to prevent deflection of cams 508, 511 during operation while not introducing excessive friction.

Pusher 204 has the general form of a horizontally oriented cylinder and is constrained by forward bearing block 506 and rear bearing block 514 so that it can only translate along its cylindrical axis, or right to left as shown in FIG. 5D. Forward stop pin 516 and rear stop pin fit through holes in pusher 204. Driving pin 522 fits through slot 521 in pusher 204 so that pusher 204 may translate a short distance left to right independently of driving pin 522. Overload spring is a compression spring and fits concentrically around pusher 204 and is compressed between forward stop pin 516 and driving pin 522. The spring constant, free length, and compressed length of overload spring 515 are determined such that the force applied to driving pin 522 is somewhat in excess of the maximum force required to press valve blade 405 into cassette 102.

Cross arm 513 is disposed generally perpendicular to pusher 204 and has a central opening the fits loosely around pusher 204 and overload spring 515. Driving pin 522 fits closely through holes on either side of cross arm 513 so that it translates horizontally with pusher 204 but can rotate independently through a small angle. At the upper and lower ends of cross arm 512 are mounted upper cam follower 519 and lower cam follower 520, which are in line with the faces of upper cam 508 and lower cam 511. Retraction spring 517 is a compression spring and fits concentrically around pusher 204 and is compressed between rear bearing block 514 and rear stop pin 518; it applies significantly lower force than overload spring 515 and has the function of keeping the cam followers 508, 511 seated against the respective cams 508, 511. Retraction spring 517 prevents the mechanism from rattling and further prevents pusher 204 from inadvertently translating to the right when no force is being applied to it.

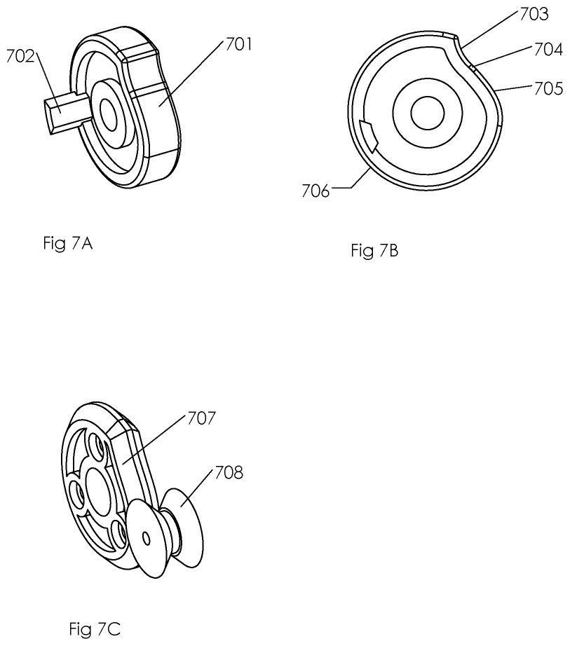

FIG. 6 is simplified, cut-away view of control mechanism 302 and cassette assembly 102. Cassette mounting component 206 is not shown, but cassette assembly 102 is shown in the position to which it would be mounted. When cassette 102 is fitted to cassette mounting component 206, the movable axis of valve blade 405 is directly in line with the movable axis of pusher 204. Further, cassette assembly 102, pusher 204, cross arm 513 and cams 508, 511 are all centered on a single vertical plane. Cams 508, 511 are shown rotated to the angle where their smallest radii are in contact with cam followers 519, 520, with contact being maintained be retraction spring 517. Overload spring 515 presses diving pin 522 to the leftmost end of slot in pusher 521.

To fully describe the action of control mechanism 302 several, several terms related to cams must be defined. Cams 508, 511 are of circular type, generally a disk-shaped component having a circumference of variable radius and rotating about a central axis. A cam follower typically is maintained in contacted with the circumference of a cam and is constrained to move generally linearly in a radial direction relative to the cam axis. A “rise” results when a cam rotates such that the cam follower contacts a greater cam radius, and thus is displaced outward, while a “return” results when a cam rotates such that the follower contacts a lesser radius, and thus is displaced inward. A “dwell” is a rotation over a constant radius portion of a cam, wherein the cam follower does not move. A cam may also be said to have “base” and a “peak,” the positions of minimum and maximum outward displacement of the cam follower respectively; there may be a dwell at either the base or peak.

Rotation of either of cams 508, 511 results in a rise or return that causes a displacement of corresponding cam follower 519, 520 and of the corresponding end of cross arm 513. Driving pin 522, at the center of cross arm 513 is displaced an average of the displacement of cam followers 519, 520 because, in the illustrated embodiment, cam followers 519, 520 are equidistant from driving pin 522 along cross arm 513. Driving pin 522 then acts through overload spring 515 to displace pusher 204. When pusher 204 is displaced sufficiently it contacts valve blade 405 and presses it into cassette 102, compressing spring 406. As valve blade 405 moves, the occlusion of flexible tube 408 is gradually removed, allowing fluid to begin to flow.

Operation of the control mechanism typically begins with cams 508, 511 both rotated to base position so that pusher 204 is in its most retracted, or leftmost, position. Cassette assembly is then fitted to cassette mounting component 206. In this state there is a gap between the rightmost end of pusher 204 and valve blade 405.

With either of cams 508, 511 held at base position, rotating the other cam from base to peak angle displaces pusher 204 to approximately close the gap, but will not move valve blade sufficiently to allow flow through flexible tube 408. Conversely, rotation of both of cams 508, 511 intermediately between base and peak angle will displace pusher 204 and does move valve blade 405 sufficiently to partially open the occlusion of flexible tube 408 and thereby allow flow.

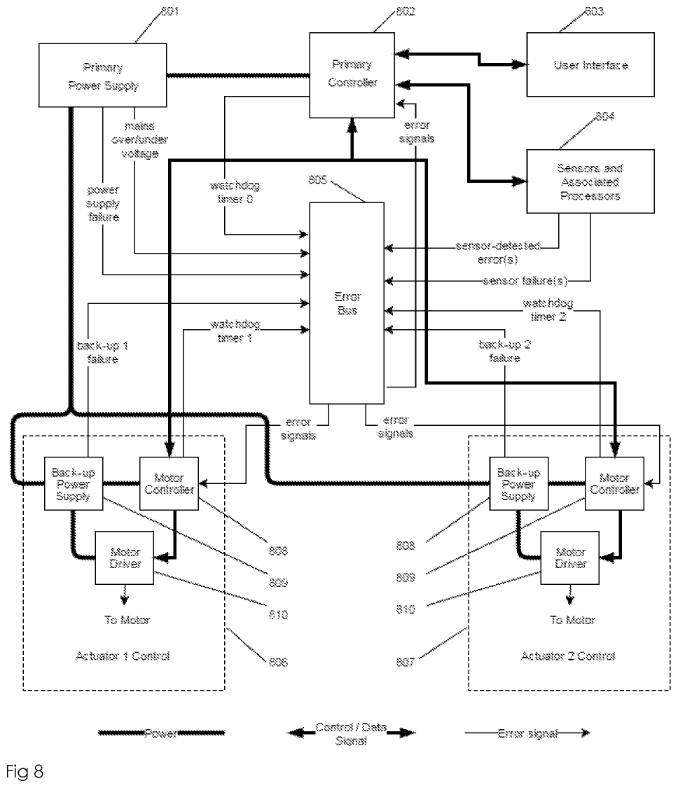

FIGS. 7A and 7B show detail of cam 508, 511. Circumferential surface 701 acts against the cam follower and consists of several tangentially connected segments. As shown the cam rotates counterclockwise for rise and clockwise for return, but this is an arbitrary distinction for purpose of illustration. Base segment 704 is a short dwell segment for maximum retraction of pusher 204. Pre-flow segment 705 is short and has a rapid rise or rapid increase in radius. Rotation of both of cams 508, 511 from base segments 704 to the end of pre-flow segments 705 displaces pusher 204 sufficiently to closely approach valve blade 405 but not to press it in. Flow control segment 706 is the longest segment is has a slow rise or slow increase in radius. Rotation of both cams 508, 511 to angles within flow control segment 706 will generally displace pusher sufficiently press valve blade 405 inward to allow flow.

Tab 702 acts as a target for a fixed optical detector to determine a home or reference angle, typically on base segment 705. For typical operation cams 508, 511 rotate bidirectionally so that pre-flow segments 705 and flow control segments 706 are employed both for rise, which starts or increases flow, and return, which stops or decreases flow. Optionally, fast-return segment 703 may be employed to stop flow. This may be advantageous for high flow rates, where either of both of cams 508, 511 are rotated to the peak angle. If it is desired to rapidly stop this flow, either of both of cams 508, 511 can rotate forward through a short angle over fast return segment 703, rather than rotate back through a large angle over flow control segment 706 and pre-flow segment 705. Base segment 704 will more quickly be reached, ensuring that flow is quickly stopped.

Overload spring 515 provides a protection feature for control mechanism 302. Normally overload spring 515 is compressed to a fix length between forward stop pin 516 and driving pin 522, with drive pin 522 seated at the end of slot in pusher 521. If an excessive force is directed against pusher 204, forcing it toward its retracted position, overload spring 515 will further compress. Driving pin 522 will not be displaced because slot in pusher 521 will move relative to it. When the excessive force is removed, pusher 204 will return to its original position, overload spring 515 will return to its original compression, and driving pin 522 will be re-seated at the end of slot in pusher 521. This protection means prevents excessive forces from being transmitted from the robust pusher 204 to more fragile and precise components of control mechanism 302. As will be shown, this protection means also allows the cassette mechanism to be removed at any time without damage to control mechanism 302.

It can be seen that control mechanism 302 provides redundancy of motors 503, 505, gear reducers and bearings 507, 512, cams 508, 511, and cam followers 519, 510. The intent is that failure of any one of these components will not cause an unsafe condition. To accomplish this intent, an electrical control system is required that detects errors and that also has certain redundant components, as will now be reviewed.

Electronic Control:

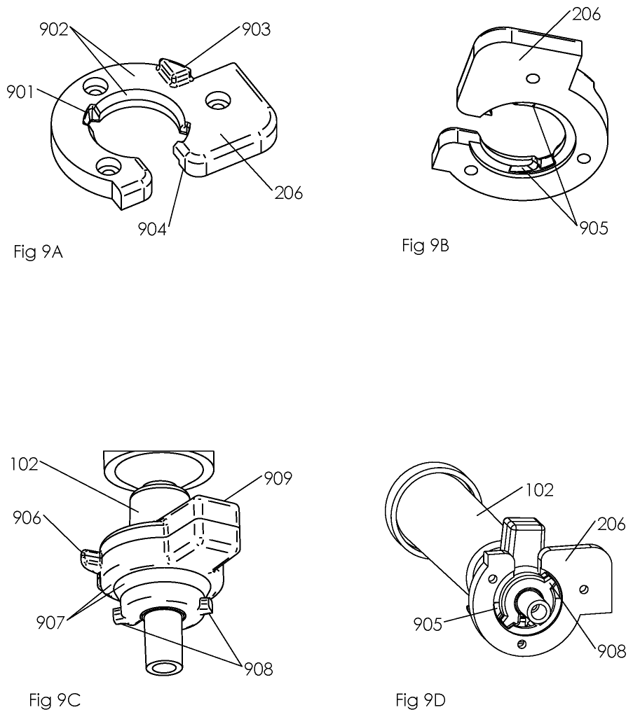

FIG. 8 is a simplified diagram of the electronic control system of IV controller 101. This is a functional diagram and does not necessarily represent the specific electronic components of the electronic control system. Primary power supply 801 receives electrical power from a mains source, battery, or both and supplies power, directly or indirectly, to all other components of the system. Primary controller 802 is a micro-controller and associated auxiliary components and performs all high level control and communications functions. User interface 803 connects to primary controller 802 and may be any arrangement of display and user input elements appropriate setting up and programming the IV controller. Sensors and associated processors 804 generally includes all sensors that monitor flow rate and the status of internal or external features of the IV controller. First actuator control 806 and second actuator control 807 are functionally equivalent subsystems, each of which includes the components motor controller 808, back-up power supply 809, and motor driver 810. First actuator control 806 is electrically connected to upper motor 503 and second actuator control 807 is electrically connected to lower motor 505.

Motor controllers 808 are micro-controllers which may have specific features for motor control applications. They may also each consist of a general purpose micro-controller connected to a second component with specific motor control features. Motor controllers 808 receive commands for motor operations from primary controller 802, perform low level motor control functions, send signals to respective motor drivers 810, and may receive feedback signals from respective motors 503, 505, other encoders or resolvers, or optical detectors 509, 510, as well as responding to error signals, as described below. Motor drivers 810 receive low-level control signals from respective motor controllers 808 and, in response, deliver controlled electrical power to respective motors 503, 505.

Back-up power supplies 809 independently store electrical power and provide power to respective motor controllers 808 and motor drivers 810 in case of loss or power from primary power supply 801. Electrical power may be stored in secondary (rechargeable) batteries or capacitors that are charged from primary power supply 801. Alternately electrical power may be stored in long-life primary batteries. In either case, each of back-up supplies 809 contains its own, independent storage component. The amount of power stored is at least adequate for respective motors 503, 505 to rotate cams 508, 511 from any position to the base position and to power respective motor controllers 808 to remain operating while that rotation takes place.

Error bus 805 routes electrical error or failure signals from various sources to primary controller 802, and both of motor controllers 808. Normally all error signals are brought to all three micro-controllers. The error signals, as shown in FIG. 8, may include error or failure of power supplies, mains power, sensors, or any of the micro-controllers.

The micro-processors of motor controllers 808 are programmed to monitor signals from the error bus and to take appropriate action to minimize patient risk. The simplest action, on detection of an error, is for the respective cam to be rotated to its base position and the respective motor driver then to be disabled. This will cause control mechanism 302 to stop any fluid flow, as previously described. More complex behaviors may be programmed based on best-practice understanding for specific medical applications. For example, a failure might be detected in first actuator control 806 or its respective motor or feedback elements while fluid is being delivered. It may be determined that the safest response is for second actuator control 807 to continue to control the fluid delivery rate while, at the same time, primary controller 802 raises an alarm to indicate that user intervention is needed. Programming of motor controllers 808 to respond to specific error signals may be fixed. Alternately, programming or program parameters may be set by commands from primary controller 802 to adapt the responses to error signals to best suit particular applications.

The combination of redundant features of control mechanism 302 and redundant electronics of actuator controls 806, 807 ensures that IV controller 101 can respond to power failures, processor failures, and other critical component failures by stopping fluid flow or taking other best-practice actions form maintaining patient safety.

Cassette Mounting Detail:

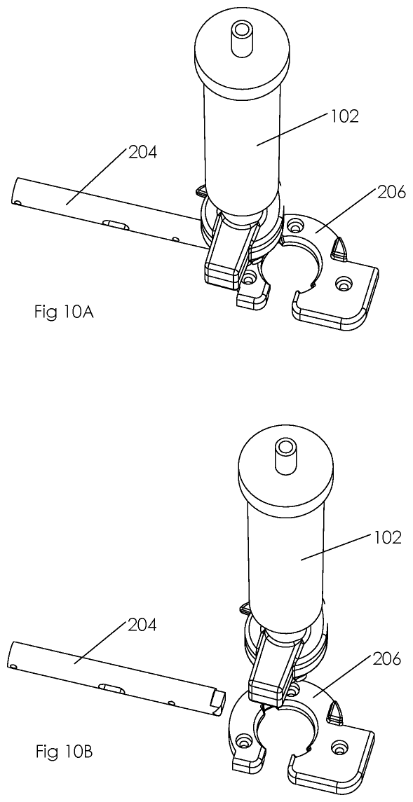

FIGS. 9A and 9B show top and bottom details respectively of cassette mounting component 206. FIG. 9C shows mounting features of cassette assembly 102, first mating surfaces 907 of cassette assembly 102 mate and contact second mating surfaces of cassette mounting component 206, aligning the axes and heights of the two components. Slot 904 of cassette mounting component 206 allows clearance for lower tube adapter 409 and a distal tube of cassette assembly 206 during insertion. Mounting tabs 908 of cassette assembly 102 pass through entry slots 901 of cassette mounting component 206 as the cassette assembly is inserted. Cassette assembly 102 is then rotated about its vertical axis, engaging mounting tabs 908 against ramp surfaces 905 of cassette mounting component 206 and so holding cassette assembly 102 firmly in place. FIG. 9D is a bottom view of the components, showing their alignment after insertion of the cassette assembly 102 after it has been inserted but before it has been rotated. Rotational alignment of cassette assembly is controlled by rotation stop 903 of cassette mounting component 206, which limits the travel of projection 909 of cassette assembly 102.

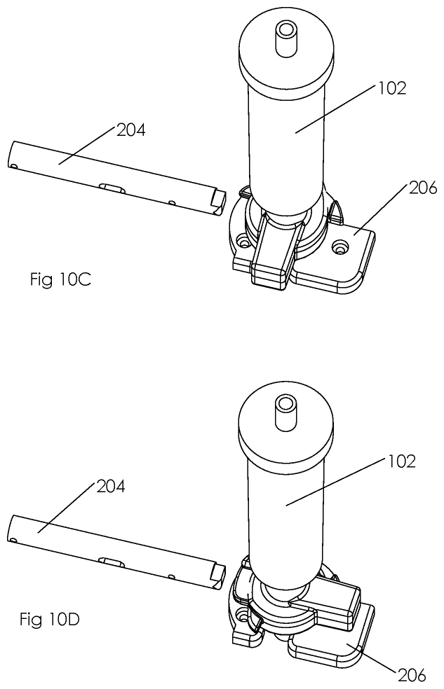

FIGS. 10A through 10D show the complete mounting sequence for cassette assembly to cassette mounting component 206. Pusher 204 is also visible to show relative orientation. In the initial position (FIG. 10A) cassette assembly 102 is above and forward of cassette mounting components. Cassette assembly 102 is then moved backward so that it is vertically aligned with cassette mounting component 206 (FIG. 10B). Cassette assembly 102 is then lowered until it contacts and is centered by cassette mounting component 206 (FIG. 10C). Finally, cassette assembly is rotated and is then held in place to cassette mounting component (FIG. 10D). As can be seen, the movable axis of valve blade 405 is then coaxial with the movable axis of pusher 204. To remove cassette assembly 102 the above steps are simply reversed.

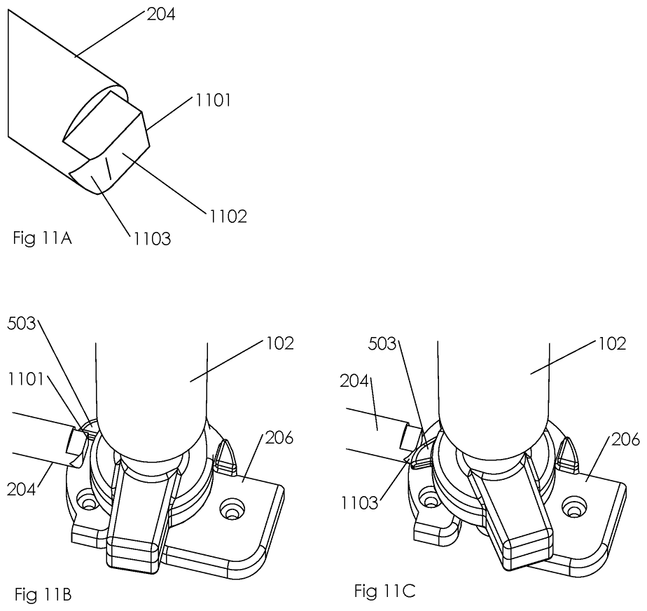

FIGS. 11A through 11C show an interlock feature that prevents unintended fluid flow under specific conditions. Pusher 204 is normally fully retracted when cassette assembly 102 is mounted. It may happen that pusher 204 remains in an extended position due to misuse or some device failure. If cassette assembly 102 is inserted and rotated into its mounted position in this instance then valve blade 405 may be forced against extended pusher 204 and pushed inward, allowing inadvertent fluid flow. This is prevented by the interlock feature as shown. FIG. 11A shows detail of the tip of pusher 204. Flat surface 1102 is the contact against valve blade 405 during normal controlled operation. It is bounded on one side by square edge 1101 and on the other side by rounded edge 1103.

Anti-rotation lug 906, as shown in FIG. 11B and FIG. 9C, projects from the side of cassette assembly 102. Anti-rotation lug 906 is positioned so that, upon insertion of cassette assembly 102 into cassette mounting component 206, it will be adjacent to the tip of pusher 204. If pusher 204 is extended, a surface of anti-rotation lug 906 will contact and be blocked by the side of pusher 204 adjacent to square edge 1101 if the user attempts to rotate cassette assembly 102 into its fully attached position. The force against pusher 204 will be mainly sideways rather than axial and will be supported by bearing blocks 506, 514, preventing sideways displacement of pusher 204. In this way cassette assembly is prevented from rotating, valve blade 405 is preventing from striking pusher 204, and thereby inadvertent fluid flow is prevented.

It is desirable that anti-rotation lug 906 does not prevent removal of cassette assembly 102 from Infusion controller assembly 101 at any time, even if pusher 204 is extended. As shown in FIG. 11C, rotation to remove cassette assembly brings anti-rotation lug 906 into contact with rounded edge 1103 of extended pusher 204. The contacting surface of anti-rotation lug 906 is tapered so that it presses obliquely against pusher 204, rather that sideways. Further, rounded edge 1103 presents a smooth, low-friction bearing surface. The resulting forces against pusher 204 are both sideways and axial. The axial force is transmitted through forward stop pin 516 to overload spring 515. Overload spring 515 compresses, as previously described, allowing pusher to retract sufficiently so that anti-rotation lug 906 can move over it. Cassette assembly 102 can thus be rotated to a position where it can be removed and where valve bladed 405 is no longer in alignment or contact with pusher 204, without causing damage to either cassette assembly 204 or control mechanism 302.

Weight Sensor:

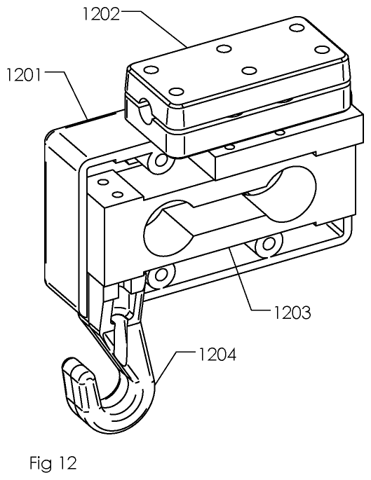

The IV Controller may employ a second sensor in addition to a drop counter to monitor fluid flow, thus providing redundant and orthogonal inputs. A weight sensor, as shown in FIG. 12 may be used to weigh the fluid container during fluid delivery and record the reduction in container weight as fluid is delivered. Enclosure 1201, shown partly removed, contains load cell 1202, of known type. For use, a bag or bottle type of container is hung from hook 1203, which is attached to the free end of load cell 1202 and extends through an opening in enclosure 1201. The other end of load cell 1202 is fixed to enclosure 1201 and to IV-pole adapter 1204, which clamps onto a bag hangar of standard type at the top of an IV pole of standard type.

Materials and Fabrication:

Certain components of the IV Controller that are exposed to high stress of potential surface damage will generally be made of metals such as aluminum, brass, titanium, austenitic (non-magnetic) stainless steel, and similar materials. These include pusher 204, pins 516, 518, and 522, and shafts and other elements of motors 503, 504, gear reducers and bearing 507, 512. All other components may be fabricated from a variety of materials including the above metals and various polymeric materials including ABS, nylon, and polyacetal. Fabrication methods for components may include machining, stamping, molding, casting, and 3D printing.

### ADDITIONAL EMBODIMENTS

The additional embodiments disclosed below are numbered for convenience; numbering does not indicate preferences as among the embodiments.

Another embodiment is similar to the first embodiment except that magnetic motors are used in the drive the flow control mechanism rather than motors of a non-magnetic type. These magnetic motors may be of known stepper, brushless servo, or brushed servo types and will generally include or be attached to reducer elements of known types to match the motor speed and torque to the requirements of the control mechanism. Feedback elements of known types may be placed at the motor, at the cam or other actuator, or in other locations to allow closed-loop control of actuator position.

Another embodiment is similar to the first embodiment except that the contact surfaces of the cams and cam followers have a V-shaped profile as shown in FIG. 7C. The cam V-profile is matched in angle to the cam follower V-profile 808. This arrangement is self-aligning. The V-profile results a rubbing action during rotation that wipes dust or grit off the surface of the cam and follower. It is important that these surfaces remain clean to maintain a smooth and predictable relationship between cam rotation and cam follower rise. The wiping action also results in friction at the line of contact. A common problem with cam followers is that they can “freeze”: wear or contamination can increase friction at the follower hub until the follower no longer rotates and instead slips along the cam surface. The increased friction between the cam and cam follower due to the V-profile contact can prevent this slipping, ensuring that the follower continues to rotate.

Another embodiment is similar to the first embodiment except that the two cams may have different profiles. For example, one cam may have a slower rise for fine adjustment and the other a faster rise for more rapid adjustment. Also, one cam may be shaped to provide motion only between the fully retracted and the working position, with the other cam shaped to provide all flow control and adjustment.

Another embodiment is similar to the first embodiment except that the cross arm component may be asymmetric, with one end longer than the other. The cam at the longer end will provide greater movement resolution while the other cam will provide greater speed, given similar cam profiles at both ends.

Another embodiment is similar to the first embodiment except that the cams rotate in opposite directions to move the pusher forward, whereas in the first embodiment the rotate in the same direction to move the pusher forward. In this embodiment the cams would generally be mirror images of each other.

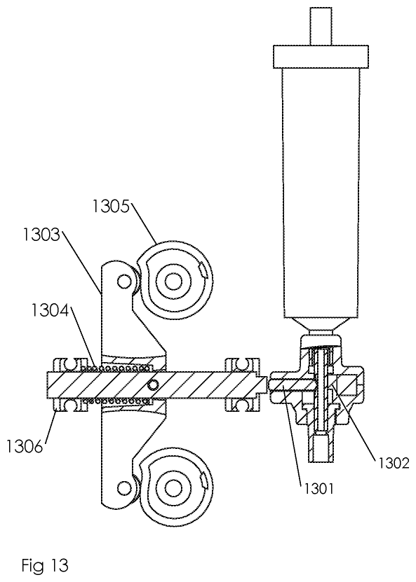

Another embodiment, shown in FIG. 13 in simplified form, is modified from the first embodiment so that no compression spring is required in the cassette. The geometries of alternate valve blade 1301 and alternate stop block 1302 in modified cassette enclosure 1303 are reversed relative to those in the first embodiment so that the valve blade is pressed in to stop flow rather than to allow flow. Actuator spring 1305 is a compression spring, compressed against alternate rear bearing block 1307, which normally acts to force modified pusher 1308 into the extended position, occluding the flexible tube 408. Alternate cross arm and cam followers 1303 are mounted in reversed orientation relative to the first embodiment, and act to retract the pusher against the force of actuator spring 1305 and thereby to allow flow. Alternate cams 1306 have a modified profile relative to the cams in the first embodiment. The geometries of the moving components in this embodiment may be such that rotation of either of cams 1306 to its base angle will allow actuator spring 1305 to fully occlude flexible tube 408, regardless of the position of the other of cams 1306.

Another embodiment is similar to the first embodiment except that motorized linear drive mechanisms are used instead of cams and cam followers the move the ends of the cross arm. Types of motorized linear drive mechanisms that may be employed include motor and lead screw combination of various types, where either the lead screw or the lead screw nut may rotate to generate linear motion. Motors of the linear piezoelectric type, or other mechanisms that create a high resolution stepped motion may also be used to generate linear motion.

A ninth embodiment is similar to the first embodiment except that pneumatic or hydraulic actuators are used instead of cams and cam followers the move the ends of the cross arm. A particular configuration of this embodiment employs a pair of flexible metal bellows, of known type, as pneumatic elements. The bellows are attached to either end of the cross arm. Air pressure in either or both bellows will push the ends of the pusher arm forward, advancing the pusher. To convert variations in air pressure to variations in displacement, rather than variations in force, the bellows can be made relatively stiff, as to act as a stiff spring, or separate stiff return springs can be attached to each end of the pusher arm. The springs must have a higher spring constant than the clamping spring in the cassette. The displacement of each pusher arm end will then be approximately proportional to the applied air pressure divided by the return spring constant, with the clamping spring having only a small effect on the displacement.

Metal bellows are preferred over a piston and cylinder arrangement for several reasons. The bellows may act intrinsically as a return spring. It can bend laterally and so may be attached to the pusher arm end and a fixed support and without pivoting joints and yet allow the pusher arm to pivot over a small angle. The metal bellows is essentially frictionless and does not have the stick-slip behavior of a sliding piston, allowing high resolution of motion in response to small changes in air pressure.

The use of pneumatic actuators may be advantageous for applications where no motors or any power electronics may be used in the vicinity of the patient. The pneumatic actuators man be connected by air lines to a pair of independent, controlled pressure sources at a distance from the patient location. The same safety features as previously described can be available given that the pressure sources operate independently and have means to monitor for malfunctions and error conditions. A loss of pressure on either line will cause one end of the pusher arm to fully retract and so stop any fluid flow. The air flow through the lines will very low compared to, for example, the air flow needed for a continuously running pneumatic motor, so the air lines can be relatively long and of small diameter without creating energy loss or reduction in accuracy.

Another embodiment is similar to the first embodiment except that type of feedback devices other than optical encoders and optical limit switches is used to measure the motion of the motors or the cam or other actuating devices. Feedback devices in this embodiment may include encoders or resolvers of either linear or rotary types, using sensing means including optical, magnetic, inductive, capacitive, or electromechanical types.

An another embodiment is similar to the first embodiment except that it employs only a single actuator that is driven by a motor of non-magnetic type. The actuator is directly coupled to the pusher element without use of a cross arm element or, alternately, the actuator is coupled to one end of the cross arm and the other end of the cross arm is held fixed or manually positioned. Another embodiment is similar to the previous embodiment except that a single pneumatic actuator, as previously described, is used in place of the non-magnetic motor.

Another embodiment is similar to the first embodiment except that the pusher is constrained in a pivoting rather than a sliding manner. Instead of forward and rear bearing blocks the pusher is rigidly connected to a single fixed pivot, this pivot being located at a distance from the desired axis of pusher motion so that the circular motion of the pusher about the pivot approximates a linear motion over the short distance required for operation. Alternately, the pusher may be connected by two link arms, each of which has one rotational axis on the pusher and one at a pivot point located at a distance from the desired axis of pusher motion.

Another embodiment is similar to the first embodiment except that the flexible tube within the cassette is replaced by an extension of the distal tube that descends from the cassette. This simplifies the fabrication and assembly of the cassette and may provide adequate performance for many applications.

Another embodiment is similar to the first embodiment except that the cassette body is reduced and the valve blade and associated compression spring are part of the controller mechanics instead of part of the disposable cassette. For use the flexible tube, or alternately the distal tube of the IV set, is positioned during cassette loading so that the tube is clamped shut by the valve blade and spring. Alternately, the valve blade may be contained in the cassette body, as in the first embodiment, but the associated spring may be a part of the controller mechanics.

Another embodiment is similar to the first embodiment except the described dual motor drive mechanism with additive or averaging coupling geometry is replaced by any other mechanism of equivalent function. For example, two motors may be coupled to what are normally considered the outputs of a differential gear mechanism of common type. The third shaft of the differential mechanism, normally considered the input, then rotates through an angle that is a fixed multiple of the sum to the rotations of the two motors. The output rotation is normally greater than the rotations of the motors so additional reducer mechanisms are required to obtain the necessary resolution and torque to operate the valve mechanism.

Another embodiment is similar to the first embodiment except that the use of gravity to provide pressure to drive fluid flow is replaced or augmented by other pressure generating means. The means may include, but are not limited to, enclosure of the fluid container in a pressurized sleeve or cuff, squeezing of the fluid container using springs or other elastic elements, or fabrication of the fluid container from an elastomeric material that is stretched during filling of the container.

### Additional Main Embodiment: Sub-Drop Detection Using an Optical Imaging System

Introduction:

A novel medical drop detector described here provides sub-drop measurement resolution as well as other valuable features. It is intended for use with a common “drip chamber” as used in medical IV tube sets, although it may be applied to any measurement of flow employing an aperture or nozzle from which drops fall. It is specifically intended to provide improved measurement and feedback at low flow rates as well as to provide positive indication of dangerous free-flow conditions and to provide other functional improvements over existing drop detection means.

### Definitions

Imager, imaging device, camera: All refer to the combination of an image sensor, a lens that focuses light onto the image sensor, electronics to rapidly determine the amount of light received by each light-sensitive element and generate a corresponding digital output, and an enclosure to prevent unwanted light from reaching the image sensor. The imager may operate with visible or infrared light and the image sensor array may be of any number of pixels. With an appropriate lens, typically having cylindrical elements, a vertically aligned, linear image sensor may be used with some limitation in functionality.

Image sensor, electronic imaging device, digital camera: An electronic light sensor device consisting of a two-dimensional array of individual light-sensitive elements or pixels. The device includes electronics to determine the intensity of light impinging on each individual pixel and to transmit this information from all or selected pixels to another device in an organized manner, either in analog or digital form. Additional electronics may control exposure time and amplification (gain), either automatically or according to signals received from an external source or device.

Light source, illuminator: Both refer to a combination of a light emitting element such as a light emitting diode (LED), one or more lens elements that direct the emitted light into a generally parallel or focused beam, and an enclosure that prevents scattering of light that does not pass through the lens elements. Also generally included, if not otherwise specified, are electronics to control and modulate the output of the light emitting element.

Imaging assembly: The combination of an optical imager with one or more illuminator, mounted as to direct light toward fluid drops being generated and to obtain an image of said light reflecting off the drops.

The optical imager and illuminator(s) are defined as functional units rather than necessarily separate physical items. They may be combined into a single physical unit, share physical components, or be combined with other mechanical, optical, or electrical components.

The optics for the optical imager and illuminator may include other elements in addition to the above described lens elements. These may include, individually or in combination and without limitation: baffles, apertures, filters, diffusers, Fresnel lenses, mirrors acting to re-direct incident or reflected light, concave mirrors acting to focus incident or reflected light, or any other optical element that supports that optical principle of this invention.

Specular reflection, specular highlight: The non-diffuse reflection of light from a smooth, mirror-like surface. The reflection may be partial or complete. “Specular reflection” refers particularly to the interaction of the light and surface while “specular highlight” refers particularly to said interaction as viewed by the eye or rendered by an optical imager.

Diffuse reflection: The scattered reflection of light from an opaque or translucent material. An opaque or translucent fluid may have both a specular reflection from its surface and a diffuse reflection from its interior.

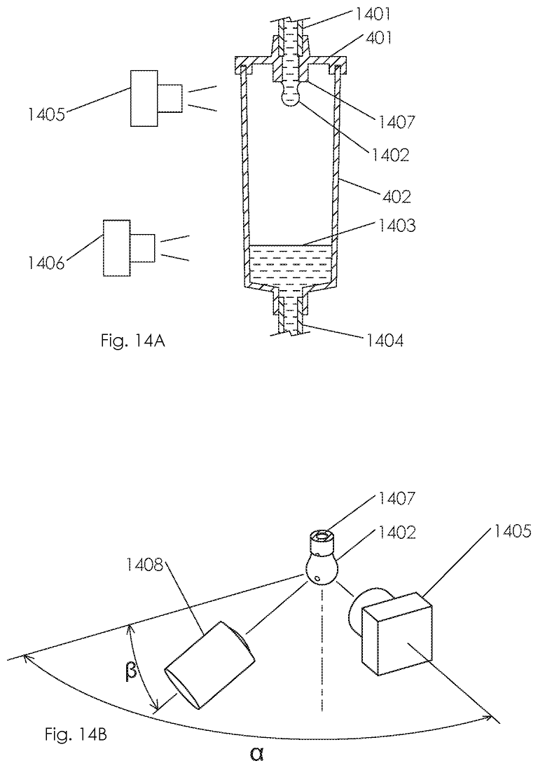

For this additional main embodiment, as shown in cross section in FIG. 14A, fluid enters through proximal tube end 1401 and forms pendant drop 1402 at the opening of nozzle in drip chamber cap 401. Upper camera 1404 is directed generally horizontally, adjacent to and directed toward pendant drop 1402 and has appropriate optics to focus at this distance.

FIG. 14B shows a more detailed view of the configuration. Optical imager 1405 and illuminated by illuminator and lens 1407 are directed toward the general location of pendant drop 1402, shown here in cut-away view, which forms at the opening of nozzle 1407. The lenses of both optical imager 1405 and illuminator 1407 are focused approximately on the near surface of pendant drop 1402.

The center of pendant drop 1402, as shown in FIG. 14B is an average or approximation because the center moves downward as the drop grows in size. Both optical imager 1405 and illuminator 1407 may be offset so that their optical axes do not intersect this approximated center. A vertical or horizontal offset may be used to obtain a more optimum position of specular highlight 1502 in image 1501 or to make best use of the available illumination and imaging fields. Distance from pendant drop 1402 to optical imager 1405 or illuminator 1407 will affect light concentration, image magnification, and the size and sharpness of the specular highlight in the camera image. All of these adjustments, as well as optimum lens types and positions, may be determined empirically, based on experimental results, or analytically.

Optical imager 1405 and illuminator and lens 1407 are spaced about an angle α about the vertical axis of the nozzle of drip chamber cap 401, typically about 45 to 135 degrees. Illuminator and lens 1407 may also positioned at an angle θ below the horizontal axis of camera 1405 and directed generally toward pendant drop 1402, the angle typically in the range of 0 to 45 degrees. One or more additional illuminators may be added, as will be further described. Camera and illuminator and lens 1407 are arranged such that the image formed by camera 1405 contains features such as specular highlights reflected from surface of pendant drop 1402. Images are obtained from camera 1405 at a rate that is a multiple of the highest expected drop rate and are processed by connected electronic or computer components to obtain a metric that varies with drop size. The metric will change as pendant drop 1402 increases in size and then revert to an initial value as the drop breaks away and falls from nozzle 1407. From the metric the number and frequency of drops is calculated, optionally to within some fraction of a drop.

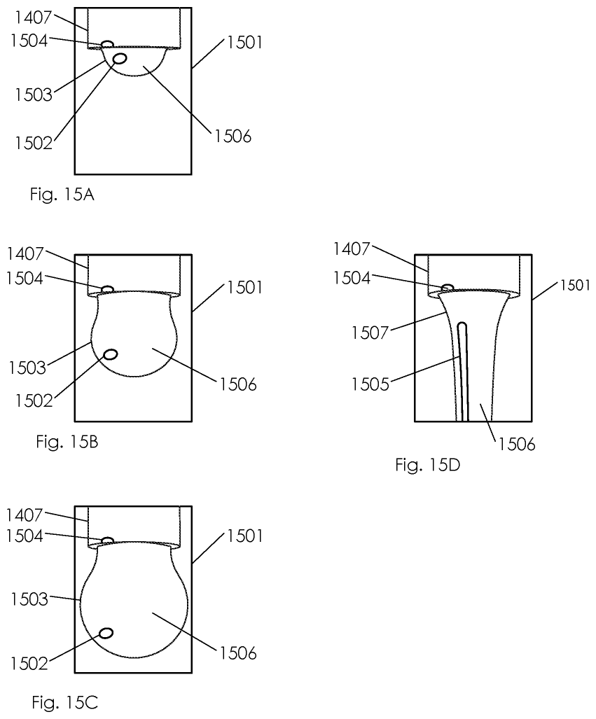

A preferred method for image processing is to detect the position of a specular highlight resulting from illumination directed toward the pendant drop and reflected toward the lens of the camera. FIG. 15A-15C show simplified views of the images obtained by the camera. In these figures, camera field-of view 1501 may represent the full field of the digital imager or a cropped portion of it. Nozzle 1407 or drip chamber cap 401 is seen at the top of the view. Typically fixed specular highlight 1504 will be seen at the edge of nozzle 1407, shown here with illumination from the left side as shown in FIG. 14A.

Pendant drop 1503 appears at the bottom of nozzle 1407; when there is fluid flow the pendant drop will grow to a maximum volume, break away, and be replaced by the next pendant drop. Specular highlight 1502 appears on the surface of pendant drop 1503 and is displaced downward as pendant drop 1503 increases in volume as seen in the progression in the volume of pendant drop 1503 from small (FIG. 15A) to medium (FIG. 15B) to large (FIG. 15C). There is also a diffuse reflection 1506 from the remainder of the drop, which will vary in brightness depending on the type of fluid but will always be less bright than specular highlight 1502. If multiple illumination sources are used then there may be multiple specular highlights on the surface of pendant drop 1503, in which case specular highlight 1502 may be considered the highlight that appears lowest in the image.

FIG. 15D illustrates the case where there a free-flow condition exists. In this case free-flow stream 1507 appears in image field-of-view 1501 instead of pendant drop 1502. Due to the shape of free-flow stream 1507, elongated specular highlight 1505 appears and can be identified through analysis of the image to detect the free-flow condition. It may be noted that the digital images shown in FIGS. 15A-15D are typically only realized as visible images in experimental work; in normal use these images are simply blocks of data stored in processor memory.

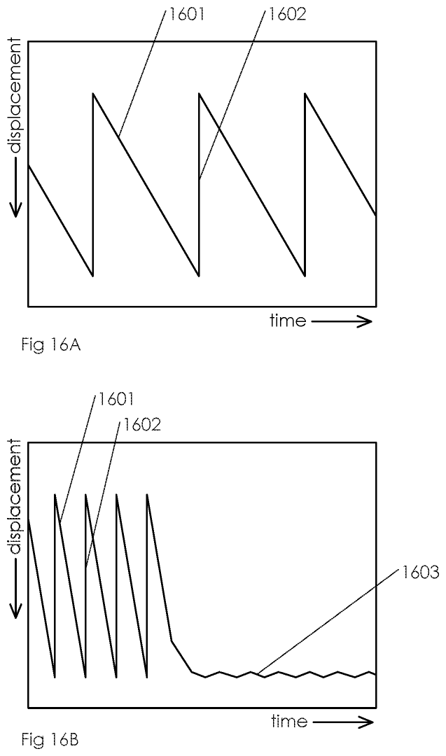

FIG. 16A shows an idealized plot of the displacement of specular highlight 1502, as determined by the imaging and processing method, over time as a series of drops form and fall away at a relatively low rate. Is this plot the displacement increases downward to relate to the actual downward displacement of the highlight. Descending line 1601 shows the increasing displacement as a drop grows. This is idealized; the actual line will typically have a non-constant slope and perturbations due to vibration and limited camera resolution. Vertical line 1602 shows the rapid return to a minimum displacement as one drop falls away and another begins to form.

FIG. 16B shows a similar idealized plot with a higher drop rate and a transition to a free flow condition. Idealized lines 1601 and 1602 again show the increasing displacement as a drop forms and the return to a minimum displacement as the drop falls away. The slope of line 1601 is greater due to the more rapid rate of drop growth. Line 1603 shows the high displacement that is maintained under a free-flow condition; this occurs because the processing algorithm always detects the greatest amount of displacement. Line 1603 is jagged because the fee-flow stream is typically unstable, producing a varying specular highlight.

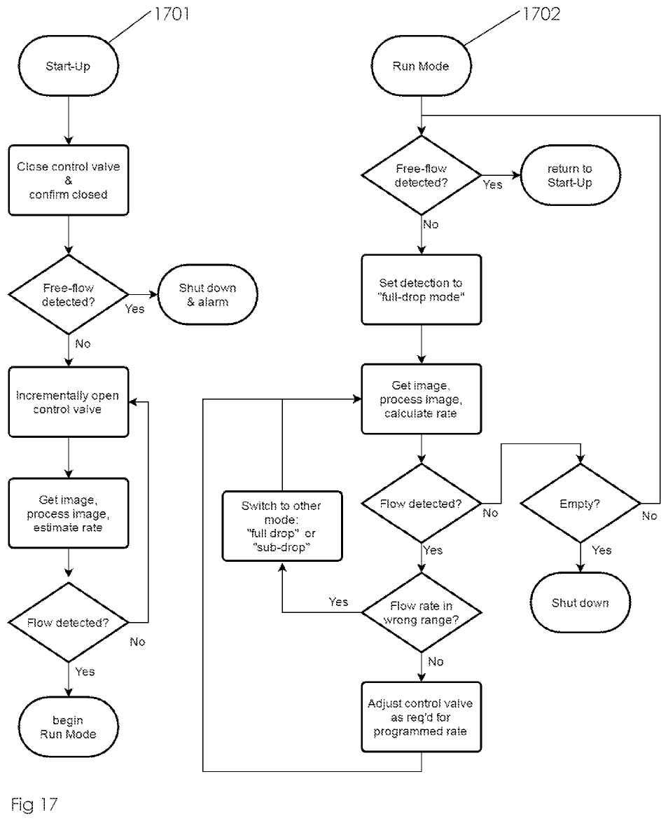

FIG. 17 is a simplified flow chart showing the operation of the digital imaging method in the context of the overall flow control system. The flow chart generalizes to any type of device that controls the rate of fluid flow, including the embodiments previously described. Start-up sequence 1701 shows the operations when flow is initiated. After closing the flow control valve a free-flow check is performed. If free flow is detected at this time it may indicate damage to system or and incorrectly loaded IV set, and operation is stopped. Otherwise operations are performed to gradually open the flow control valve until the imaging system detects the beginning of flow. The operation then switches to the run mode.

Run mode sequence 1702 also includes a regularly performed free-flow checked; if free-flow occurs start-up sequence 1701 is re-initiated to fully stop and then controllably restart the flow. The processing method is next set to a default full-drop mode, used at higher flow rates. For the remainder of fluid deliver the sequence will typically remain within a control loop where images are processed to determine the rate of fluid flow and the control valve is adjusted as required to maintain the desired flow rate. The process may switch between full-drop mode and sub-drop mode depending on the measured and desired rates of fluid flow; these modes are further discussed below. If the measured flow rate drops to zero then additional processing is preformed to determine whether the fluid container is empty, in which case the run mode sequence 1702 is completed.

Background:

A drip chamber, as shown in FIG. 14A, is a standard device employed to set and monitor fluid flow. In a basic configuration, flow is controlled by a manually adjustable clamp fitted to the tubing below the drip chamber. The user watches the drip chamber while slowly opening and adjusting the clamp. The flow rate is determined by the number of drops that fall from the nozzle or aperture in a fixed time interval. Typical drip chambers have a nominal drop size of 0.1 mL, although other sizes are commonly available. If, for example, ten drops are observed falling in one minute then the flow rate is about 1 mL/min.

Various means have been developed to automate counting of drops, either to provide easier manual control or to provide feedback for an automated flow control system. The most common method is to place a light source on one side of the drip chamber and a photo-sensor on the opposite side. A falling drop will momentarily interrupt the light path, generating an electronic signal. Common issues with this method include false counts due to splash-back from the fluid pool at the bottom of the chamber, missed counts due to misalignment causing drops to fall outside the light path, and general unreliability due light transmission being reduced by splashes or condensation on the inside wall of the chamber. A further complication is that translucent and opaque fluid block the light to varying degrees but transparent droplets refract the light, causing a confusing variety of signals from the photo-sensor. Also, ambient light such as sunlight may produce unwanted signals from the photo-sensor.

A critical problem with this type of drop detector is that it cannot reliably detect a free-flow condition, in which there is a continuous stream of fluid instead of individual drops. This condition, which can easily cause overdoses, is hard to differentiate from zero flow because the optical detector is basically a differential device, producing a pulse when the light intensity changes. In both the zero flow and free-flow conditions there is little or no intensity change. Light levels will vary according to the conditions of the drip chamber walls and the fluid opacity, so specific intensities are not meaningful for detection.

By various method, optical drop detectors have been made to work reliably under most conditions. One method is to automatically adjust the intensity of the light source or the gain of the sensor to compensate for variations in light transmission. However, this may further reduce the ability to a detect free-flow condition because the actual light transmission can no longer be determined.

Other sensor types have been developed, but none has shown sufficient advantage to replace optical detectors. Developments by Kamen et al (included in the information disclosure filed with this application) attempt to improve reliability and detect free-flow but appear only to be practical for transparent fluids; their functionality is lost with common lipid-based medical fluids, which are opaque. A development by Hungerford et al (also part of the information disclosure) attempted to use optical means to determine the size of the fluid drop for improved flow measurement accuracy; it appears that it this also is intended primarily for transparent fluids. This development will be discussed in further detail because it has superficial but ultimately immaterial points of similarity with the present invention.

A specific limitation of existing drop detectors is that their resolution is limited to a full drop, most commonly 0.1 mL. For many medical fluid delivery applications it is desired to deliver fluid at low rates where the time between drops may range from 5 seconds to 60 seconds or more. This makes timely measurement of flow rates very difficult. For a manually controlled system a nurse may have to watch for several minutes to ensure a correct flow setting. For an automated system there will be extensive delays in the feedback loop used to control the flow setting. In control system theory it is known that errors increase exponentially with feedback delay times; systems can easily become inaccurate or unstable under conditions with long delays. Delays will negatively affect the maintenance of a desired flow rate but will have an even greater effect when flow is started or the flow rate is changed. In these cases the flow control setting can only be changed very slowly, otherwise the setting change may cause the flow rate to greatly overshoot the desired change. In medical fluid delivery this may cause a dangerous overdose or under-dose of a drug.

### Operation of Additional Main Embodiment

As shown in FIG. 14A, one or more optical imagers 1405, 1406 may be used to monitor drop formation as well as other aspects of the drip chamber and contained fluid. FIG. 14B shows an arrangement of digital optical imager 1405 (upper camera) and illuminator 1408 (light source), mounted in fixed positions relative to a component such as medical drip chamber nozzle 1407, from which fluids drops form, hang, grow, and then break away in a repeating cycle. Illuminator 1408 is positioned to direct a beam of light on to the hanging, or pendant, drop 1402. Optical imager 1405 is positioned from a different angle to view pendant drop 1402 by light reflected from illuminator 1408.

Images obtained from optical imager 1405 will contain a specular highlight 1502 that is a reflection of the light source on the convex, partially reflecting surface of pendant drop 1402. The location of this highlight will depend on the size of the drop. As the drop grows highlight will generally move downward in the image. Note that the actual orientation of optical imager 1405 is arbitrary; the directions “up”, “top”, “down”, “bottom”, “left”, and “right” are used here as the natural orientation that one would apply to an image; nozzle 1407 is at the top of the image and pendant drop 1503 grows downward toward the bottom of the image.

As shown in FIGS. 15A-15C, the position of highlight 1502 along the vertical (up-down) axis within image 1501 is used as an indication of the size of pendant drop 1503. This distance from the top of image 1501 may be taken as a positive value; another reference, such as the vertical location in the image of fixed specular highlight 1504 at the tip of nozzle 1407, may alternately be used. This value will generally increase as the drop grows and then rapidly return to a smaller value as the drop breaks away and a new drop begins to form, producing a generally saw-tooth type of signal.

An idealized view of this signal is shown if FIG. 16A where line 1601 shows and increase in displacement of highlight 1504 and line 1602 shows this displacement returning to a minimum value as a drop breaks away from nozzle 1407. In actuality, the slope of the increasing signal will not be constant; there is a non-linear relationship between the drop volume and the highlight position. The drop has a complex, dynamically changing shape; initially the length of the drop increases more rapidly than its diameter but shortly before the drop breaks away its diameter increase more rapidly. Environmental vibration may also create waves or momentary distortions in the drop surface.

The images obtained from the camera may be processed by appropriate software or firmware. Processing generally involves at least five tasks including: locating the specular highlight within the image, filtering and other processing to remove noise and optionally to linearize the output over time, detecting of presence of flow, detecting full drops (i.e. drops breaking away from the nozzle), and estimating sub-drop flows during periods between the break-away of drops.

In general, the results of this processing will be used immediately for control purposes. Therefore, all processing must take place in real time. It may be useful to “look back” over previously collected data for reporting or optimization purposes, but for control purposes it is important to minimize delay. For example, an estimate of sub-drop flow is only useful up to the time a drop breaks away and completes a full drop of flow.

Experimental Results:

An apparatus was constructed to experimentally test imaging of drop formation. A 3D-printed holder for a drip chamber was mounted to a vertical pole, with a camera and illuminators mounted adjacently. The camera was a model M7 from OpenMV LLC (openmv.io), a VGA resolution camera with machine vision functions that can be programed using the MicroPython language. For these experiments it was equipped with a visible-light blocking filter (Edmund Optics part #43-954, cut to size). Software from OpenMV was installed on a Windows? PC to interface with the camera, provide a programming environment, and display and save images and video. Two illuminators were constructed, also by 3D printing. Each contained an 850 nm infrared LED and a 10 mm focal length, 8 mm diameter acrylic lens. The lens was positioned approximately 15 mm from the LED, giving a partially focused illumination beam.

For these experiments the camera was positioned approximately 25 mm from the drop axis of the drip chamber, oriented horizontally, and positioned so that the tip of the drip nozzle and the maximum size hanging drop were visible in the frame. Two illuminators were installed, one aligned horizontally and at the same height as the camera and the other mounted lower and tilted upward at about 35 degrees to be directed at the drop forming area. For the experiments described here both illuminators were turned on continuously, each operating at approximated 30 mA.

A MicroPython program was written to capture images and save them to a MJPG video file. Images were captured for 15 seconds at 20 frames per second, for a total of 300 frames in each test file. Exposure and gain were set manually to provide consistent brightness levels in the images. Images were captured at low resolution and then cropped for a final 40×100 pixels. The camera was mounted sideways from its normal orientation so that the 40 rows became columns and the 100 columns became rows. This gave a narrow rectangular frame on the drop forming area.

Video was captured with a variety of drop rates as well as free (continuous) flow. The individual video files were processed using a Python 3.6 program using the OpenCV video processing library and the Pyzo development environment.

Each video was processed frame by frame in the following sequence of steps: Read frame in from file. Remove extraneous data and rotate frame to vertical. Copy with thresholding such that all pixels above a certain value became white and all other pixels became black, with a threshold level selected to preserve the specular highlight(s) and remove all other features in the image. Analyze the thresholded copy to find the lowest white pixel in the image, locating the position of the lowest specular highlight, and save this position to an array, which holds this information for all frames.

Once this was completed the position data was further processed in several steps to filter noise, provide partial linearization, and detect full drop and (estimated) sub-drop intervals. The original and threshold frames were also combined and saved to a new video file for further viewing.

Experimental results were that full drops could be clearly detected both for slow and moderately fast drop rates. At faster drop rates there was aliasing due to the relatively low frame-per-second rate. This caused occasional errors in full drop detection. These errors may be eliminated either using a faster frame rate or a more advanced processing algorithm.

Estimation of sub-drop rates was successful but had limitations. This estimation depends on prediction of the current fraction of a drop of flow based on previous drop cycles. The 15 second video capture period did not provide adequate time to develop a basis for this estimation. Despite this, the estimation was typically accurate to within ¼ of a drop. Further, the sub-drop estimates provided verification of continuing flow, typically detecting new drop growth within 1/10 of a drop after the previous full drop had fallen away.

Free Flow Detection: Free-flow or streaming flow can be robustly detected with the present invention. A specific image “signature” appears during free-flow that can be easily and reliably analyzed. As shown if FIG. 15D, elongated specular highlight 1505 has the general form of a narrow vertical band in image 1501; this is a reflection from the vertical free-flow stream 1507 exiting the nozzle.

A test was performed in which flow was initially at the highest rate at which drop regularly appeared. After several sections the clamp on the tube set was fully opened, allowing free or streaming flow. FIG. 16B shows an idealized highlight displacement signal for this test, with free-flow line 1603 showing maximum displacement because elongated highlight 1505 extends to the bottom edge of image 1501. The appearance of highlight 1505 as a vertical band appears most prominently if both the optical imager 1405 and illuminator 1408 are positioned with approximately horizontal orientation and at roughly the same height. If illuminator 1408 is mounted lower and aimed upward then the bright band appears shorter and tends to be curved, but can still be detected.

Several alternatives are available for illumination and imaging for free-flow detection, including: Use only a horizontally aligned illuminator with some loss in drop growth resolution. Use only an upwardly angled illuminator, with less definition of the free-flow signature. Use both horizontal and upwardly angled illuminators, with both turned on for all images. Use an upwardly angled illuminator for normal drop detection and separately image with a horizontal illuminator for free-flow detection.

It may be noted that whenever a positive drop detection occurs it is generally not necessary to perform free-flow detection. Only if no flow or an inconsistent flow signal is detected is there the potential for an actual free-flow condition that should be detected. A simple algorithm was tested to detect free flow from the raw images. The image was first cropped to isolate the portion where the bright band could occur. Then each row in the cropped section was analyzed to determine whether a) it had at least one pixel above a threshold level and b) pixels at both ends of the row were darker than the brightest pixel by a second threshold amount. If both criteria were found for some minimum number of rows then free-flow detection is considered positive.

Test results showed positive detection for all visually observed free-flow incidents. Occasional momentary free-flow detection occurred at other times, possibly in indicating unstable flow conditions. These potentially false detections may be eliminated by requiring that the free-fall condition be detected for some minimum period of time before it is considered a positive detection. Alternately, the free-flow signature could be more analyzed in more detail. As example, the algorithm could inspect for consistency in brightness and horizontal position between the peak brightness pixels in successive rows.

Both the optical arrangement and the detection algorithm may be further optimized, but the basis for robust free-flow detection is contained with the present invention.

Fluid Type Detection:

The arrangement of optical imager and illuminators described here may be used with both transparent and opaque fluids, an advantage over some other drop detection developments. Specular highlight 1502 or elongated specular highlight 1505 will be similar in either case. Diffuse reflection area 1506 over the remainder of pendant drop 1504 will be relatively dark for transparent fluids and brighter (although still darker than the specular highlights) for white opaque fluids. Darker-colored fluids will, with appropriate illumination, produce lower levels of diffuse reflection than clear liquids.

Analysis of the relative brightness of the specular highlight 1502 and diffuse reflection area 1505 within image 1501 may be used to determine the type of fluid in the device. Most medical fluids are either clear saline aqueous solutions or opaque white lipid solutions. Lipids are composed of microscopic oil or fatty drops in an aqueous base, typically about 80% water by volume. Drop sizes are generally consistent within each group of fluids, but vary between the two groups because the lipid solutions have different viscosity, density, and surface tension than the aqueous solutions. Detection of the fluid type allows corrections to be made to the expected volume per drop, allowing for more accurate control or reporting of delivered volumes and flow rates.

The present embodiment can determine the type of fluid by the relative brightness of the specular reflections and the diffuse reflections from each (or any) drop. This may be used in the absence of any other means of determining the fluid type and can be used to improve the accuracy of control or reporting of delivered volumes and flow rates. If there is another active means for determining fluid type, such as an internal drug library, the present invention can be used to verify that identification of a fluid as aqueous or lipid by said other means is in fact correct.

Physical Details of Optical Imager and Illuminator.

Optical imager 1405 is essentially a digital camera consisting of a focusing lens, digital imager and associated electronics, and a light-tight enclosure. It may use visible or infrared light; in one preferred non-limiting embodiment it has high sensitivity to wavelengths around 850-nm and filtering to block visible light. The image sensor resolution may be 100×40 pixels or less. Most commercial image sensors have higher resolutions but can operate at reduced resolutions either by using only part of the sensor area or by “binning,” a process in which signals from several adjacent pixels are combined into a single output. These methods can be used advantageously to reduce power consumption, shorten exposure times, and control the degree of magnification.

Optical imager 1405 will normally be placed with its lens close to the wall of the drip chamber body 402. The exact position and choice of lens will depend on the size of the image sensor. For example, a common size for low-cost image sensors is approximately 4 mm×3 mm. The vertical length of pendant drop 1503, just before break-away, plus the tip of nozzle 1407, is about 12 mm. If the 4 mm dimension is aligned vertically then the minimum 0.333 magnification is required; typically only a portion of the imager area at about a 0.20 magnification would be used. A standard lens for this sensor size is 3.6 mm focal length. Distances can be a using the “thin lens” formula

$$M = \frac{{d}_{i}}{f} - 1 which rearranges to {d}_{i} = f * ( 1 + M )$$

where M is magnification, f is focal length, and d is the lens-to-image plane distance. Using a magnification of 0.20 this gives a value of 4.3 mm for dᵢ. The lens to subject distance is then calculated as

$${d}_{o} = \frac{{d}_{i}}{M}$$

giving dₒ equal to 21.6 mm. These values are only approximate for a real “thick” lens but it is evident that the camera placement will be close to the forming drops but outside the wall of the drip chamber.

It is advantageous if the lens has a relatively larger aperture (in camera terminology, a small f-stop number) to provide reduced depth-of-field. The advantage is that, with the camera and lens positioned to focus on the fluid drop, the near wall of the drip chamber will be far out of focus. Any fluid droplets or other material on this wall will be rendered as soft, unfocused bright or dark areas rather than as focused features that might interfere with image processing.

Conversely, the lens does not need to be of high optical quality. The desired image content has very little detail and does not need to be rendered with great sharpness. With monochromatic illumination it is unnecessary for the lens to be corrected for chromatic aberration. It is important that the optical system does not have excessive flare, unfocused light reaching the image sensor that can obscure the desired image content.

Illuminator 1408 typically consists of an LED and a focusing lens in a small housing. The focusing lens concentrates the light from the LED, directing most of it toward the area of the pendant drop 1503. In a preferred, non-limiting embodiment the LED output is in the infrared range of 850 nm-940 nm. Electronic controls are provided to switch the LED on and off, as will be discussed in detail below.

One or more of illuminator 1408 is placed close to the wall of drip chamber body 402, reducing overall device size and to maximizing light concentration. The lenses for these illuminators have preferably a relatively large aperture to maximize light concentration and to provide focused light in the vicinity of pendant drop 1503 but unfocused light passing through the wall of drip chamber body 402.

The drip chamber is of a standard type, as used in medical drug delivery practice. It is a clear plastic cylinder, usually tapered, with an inlet at one end and an outlet at the other. The inlet is connected directly to an interior nozzle; the nozzle geometry has been determined to produce drops of a specified volume. In use the drip chamber is aligned vertically, with the inlet at the top. The inlet is connected to an input tube, bag, or bottle and the outlet to an output tube. A small pool of fluid at the bottom of the drip chamber acts to maintain a fixed air volume within the chamber. The flow rate or drop rate is typically controlled by an adjustable clamp fitted to the output tube below the drip chamber.

The drip chamber is positioned using a mount or clip that holds it in a vertically aligned orientation and at a fixed height relative to the image sensor and illuminator components. The drip chamber may also be part of a cassette, cartridge, or other removable assembly that mounts to a fixed location on a device that includes the image sensor and illuminator components.

The drop forming below the nozzle has a partially reflective surface. This surface generally has a continuous convex shape except in the region closest to the nozzle tip where it may be partially concave. A simple parallel or focused beam of light falling on the convex surface will produce a single specular highlight as seen from a particular viewing or camera angle. The location of the highlight is determined by the reflectance law: angle of incidence equals angle of reflectance. On a curved surface the angles are measured from the normal vector to any point on the surface. The size of the specular highlight in the camera image depends on the illumination source and optics and the camera optics.

The optical paths of either the illuminator(s) or camera may be folded using one or more mirrors or prisms to provide a more compact configuration. For example, the camera may be directed upwards and a planar mirror placed above it at an angle. Light reflected approximately horizontally from the hanging drop will be reflected by the mirror downward towards the camera. The mirror may be placed between the drip chamber and the camera lens or between the lens and the image sensor. Alternately, a concave mirror may be used to provide by optical path folding and focusing. The same arrangement may be used for a separate photo-sensor. The same arrangement may be used for an illuminator except that the light will travel upward from the illuminator and then be reflected approximately horizontally or at an upward angle toward the drop area.

A separate photo-sensor may be employed to detect drops at high drop rates or as a backup to the camera. The photo-sensor may be placed adjacent to the camera or opposed to it (i.e. located approximately 180 degrees from the camera around the drip nozzle axis) and directed toward the drop area. A lens or other optical element may be used to direct reflected light from the drop (or a portion of the drop) toward the photos-sensor while tending to block any stray light. The light source for use with this photo-sensor may be the same illuminator(s) used with the camera or an independent light source. The photo-sensor will not resolve specular versus diffuse reflection or the position of a specular highlight, but only an overall brightness measurement which is sufficient to determine the momentary presence of absence of a hanging drop.

The separate photo-sensor will be sufficient to detect full drops forming and breaking away from the nozzle, although it will not reliably provide sub-drop resolution. At high drop rates this information is sufficient and can allow the camera to not be used. This acts to reduce power requirements, both for the camera and the processor since the amount of processing required to analyze a single analog input is less than that required for camera images. Also, the illuminator may be switched on for a shorter period for each reading, typically less than 1 millisecond, further conserving power. Further, camera settings do not need to be changed between any slow and fast drop rate modes if the camera is only used for slow drop rates; with some cameras this will simplify operation. In case of some failure of the camera or its associated optical system the separate photo-sensor can act as a backup so that flow measurement and control can still be maintained, although resolution will be limited to full drops.

The separate photo-sensor may also be used to detect whether a fluid is an aqueous or lipid solution. The overall reflected brightness from a drop of opaque white lipid solution is significantly greater than that of an aqueous solution and the difference in output signal level can easily be discerned by simple processing.

In a typical measuring system the illuminator, camera, and any other elements will be located within a single enclosure. A recess on the enclosure will be provided for insertion of the drip chamber. The optical elements will be directed into this recess. In one possible non-limiting arrangement the illuminator(s) will be at the back surface of the recess, pointing forward. The camera and optional photo-sensor will on a side surface of the recess, facing either right or left. The recess surfaces may be fabricated of a dark, light absorbing material. This arrangement will act to minimize any stray or ambient light that reaches the camera or optional photo-sensor. Also, light from any secondary reflections from the hanging drop or the recess surfaces will be minimized. This arrangement is only typical and many other arrangements are possible.

Illumination and Exposure Time Issues:

To enable meaningful processing and evaluation of images obtained with this invention it is important that illumination and exposure be consistent and that the specular highlights in the image be clearly discernible. A number of factors must be considered for this purpose.

Many image sensors provide automatic gain and exposure control, which must be disabled to provide consistent intensity levels in the images. Exposure and gain will instead be controlled by signals from an external processor and may remain fixed be adjusted to compensate for changes in light transmission through the drip chamber wall or changes in ambient light.

Any ambient light such as room light or sunlight that impinges on the image sensor may negatively affect the evaluation of the images. Use of infrared illumination in combination with a visible-light blocking filter on the camera will minimize the amount of ambient light reaching the sensor. Fluorescent and LED room lights produce very little output in the near-infrared range to which the sensor is sensitive. However sunlight and incandescent light bulb illumination may have significant intensity within this range.

The image sensor samples impinging light for a programmable amount of time (exposure time), typically in the range of 1/30 to 1/100 of a second. Some image sensors will allow shorter exposure times. The illuminator may be turned on only during the exposure to reduce energy use and light source heating. Further, the illuminator may be turned on for only a fraction of the exposure time. LED illuminators can produce high light intensities without overheating or using excessive power using high current levels for short time intervals. The exposure at each pixel will be determined as

total illumination=exposure time*ambient light intensity+illuminator on time*illuminator intensity

It can be seen that the effect of ambient illumination will be minimized if the overall exposure time in reduced so that it is equal to or only slightly greater than the illuminator on time. A limitation is that most image sensors employ a “rolling shutter”: Pixels are “read-out” one row at a time so that image acquisition at the bottom of the image occurs later than at the top of the frame. Depending on the exposure time, the read-out rate, and the number of rows being used, the top rows may be read-out before exposure even begins for the bottom rows. For even illumination of the resulting image the illuminator must be on for the entire exposure time on all rows.

The image of the forming drops, and particularly the area on the drops where specular highlights occur can be framed within a narrow vertical rectangle. The image sensor may be rotated so that its rows of pixels become columns relative to the actual drop orientation; in effect the camera is turned on its side. This reduces the number of image sensor rows that must be read-out and so reduces the overall imaging time. By adjusting the exposure time in conjunction with the reduction of sensor rows employed, the required illuminator on-time may be reduced. This will minimize the effects of ambient light and also minimize any motion related blur in the image.

For example. but not limitation, a common sensor specification is a maximum 120 frames per second at 240 pixel vertical resolution (i.e. 240 rows). This gives a maximum acquisition of 0.0083 seconds for each frame and a read-out time for each row of 0.0083/240=34.72 μs (microsecond). If only 30 rows are actually used then the acquisition time will be 30*34.72 μs=1.04 ms (millisecond). The exposure time can be set to 1 ms (assuming an adequate illumination level); each successive row is exposed for 1 msec beginning 34.72 μs after exposure begins on the previous row. The total time to expose and read-out the frame is thus no more than (1 ms+30 rows*34.72 μs/row)=2.04 ms. A safe illuminator on-time may be about 3.0 ms; at an expected maximum of 30 frames per second this give about a 10% duty cycle; LED output can be high without overheating or using excessive power. The exposure time for each row is only 1.0 ms, so sensitivity to ambient light will be low. In this way the low resolution requirement of this invention allows a common, low-cost image sensor to be used while approximating the timing performance of more expensive sensor types.

Certain image sensors have a “global shutter” in which all pixels are essentially read-out simultaneously. In this case the exposure time and illuminator on-time may both be in the range of 1 ms or less. This short exposure will minimize any effect on the images due to ambient light. This sensor type has previously been more expensive and required more power to operate but “global shutter” sensors with lower costs and power usage are being developed, particularly for IoT applications, which may be applied to use in this invention.

An alternative technique for reducing the effects of ambient illumination is a technique known as background subtraction. Two images are obtained in rapid succession, one with the illuminator on and one with it off. The pixel values obtained in the “off” image are subtracted from those in the “on” image. This mostly removes the portion of the image due to the ambient light. This technique can be very effective but has several limitations. First, the time between the images must be short relative to any subject motion that occurs, otherwise there may be artifacts in the resulting image. Second, signal noise in the original images may also produce artifacts in the resulting image. Third, this technique increases power usage and reduces the maximum effective frame rate for the camera and requires additional processing.

The first and second limitations may be mostly overcome by using a slightly more involved operation than simple subtraction to reduce or eliminate artifacts. Each resulting pixel value is calculated as the lesser of the “on” image value and the subtracted value; this eliminates most high-brightness artifacts. The third limitation may be partially overcome using an adaptive process. At regular intervals an image is generated using the background subtraction technique. This image is compared to the illuminator “on” image, effectively by performing a second subtraction and summing all the pixel values. If the difference between the two is greater than a predetermined threshold then use of the background subtraction technique is continued. If the difference is below the threshold then images are obtained simply with the illuminator on and are used without background subtraction processing.

Another technique that may be applied to reducing effects of ambient illumination is to place a polarizing filter in the camera light path. Light reflected at an angle from a non-metallic surface will be polarized to a degree that depends on the angle and the material. The angle of reflection will vary somewhat as the drop grows but is mostly fixed by the positions of the illuminator and camera. A polarizing filter in the camera light path can be rotated so that most of this polarized light from the specular highlight passes through to the sensor. Ambient illumination will generally be non-polarized or have other polarization rotations and so will be attenuated by the polarizing filter. The use of a polarizing filter may also be advantageous for use with lipid-based fluids as it may be used to increase contrast between the polarized specular reflections from the surface and the non-polarized diffuse reflections from the interior of the drop.

A movable cover or door may also be provided that fits over or around the drip chamber so that it is protected from ambient or stray light. This cover may be fabricated from a material that blocks infrared light while allowing the passage of visible light. This would allow drop formation to be viewed by the user while preventing ambient or stray light from reaching the image sensor or an optional photo-sensor.

Integration with an Automated Flow Controller:

The flow rate measurement method described here may integrated with the previously described flow rate control system but may also be used independently or with other types of flow producing or flow controlling systems. The control of fluid flow may be accomplished by methods including, but not limited to: a pumping system with adjustable speed or displacement to provide variable flow; a source of pressurization, such as the weight of a column of water, and an automatically adjustable valve that varies resistance to fluid flow.

FIG. 17, showing the previously described simplified flowchart, includes references to “full drop” and “sub-drop” detection modes. Flow rates may be divided into fast and slow regimes. At higher flow rates, typically those resulting in a drop rate faster than about 1-0.3 drops per second, there is no need for sub-drop measurements. At these rates adequate feedback is supplied by full drop measurements. It may also be preferred to use higher frame rates when in this regime to minimize aliasing and the potential for missed drops. Performing only full-drop processing at higher frame rates minimizes the overall processing overhead.

At lower flow rates, with corresponding drop rates slower than about 1-0.3 drops per second, the sub-drop and flow detection processes become important to provide adequate feedback for fast and stable closed loop control. In this regime lower frame rates may be used to reduce processing overhead. When in “Run Mode” the control process may determine after each camera image is analyzed, or at other repeated intervals, whether the current flow rate is within the defined fast or slow rate regime. If the current flow rate regime does not match the active detection mode then the detection mode can immediately be switched without loss of data.

The flowchart shown in FIG. 17 is intended only for illustration purposes and does not fully represent the processes required for a real control system, particularly a real control system that is intended for medical or other critical functions.

Application of a Second Camera:

As shown in FIG. 14B, second optical imager 1406 may be positioned adjacent to the lower part of drip chamber body 402 and may be provided with any appropriate illumination. Second imager 1406 is aligned horizontally and directed generally toward the meniscus of fluid pool 1403 at the bottom of the drip chamber body 402. The meniscus can be identified in digital images obtained from second imager 1406, typically by finding locations in each column of a digital image that have abrupt changes in brightness and then correlating these locations over all the columns to determine the height of the meniscus (or depth of the fluid pool). This measurement serves two purposes: Feedback can be provided when fluid is first filled into the drip chamber to create the fluid pool, assisting the user or allowing this operation to be performed automatically. Also, the overall system may be programmed to stop flow when the meniscus drops to a certain level; this allows an empty fluid container to be replaced without needing to re-prime the IV set or refill the drip chamber. An optical imager placed in this location may also be used to obtain images of markings or codes placed on the drip chamber; these may include alignment marks to verify correct positioning of the drip chamber or cassette assembly or specific identifiers such as bar codes.

### Alternative and Additional Embodiments

One skilled in the art will understand that the flow rate measurement method described here can be used in various configurations, including but not limited to: The sub-drop counting functionality, free-flow detection functionality, and fluid-type detection functionality as described above may be employed independently or in any combination. The camera and one or more illuminators as described may provide drop counting, sub-drop counting, free-flow detection, and fluid-type detection functions, singly or in any combination. The combination of one or more illuminators, a camera, and a separate photo-sensor may be used to provide drop counting, sub-drop counting, free-flow detection, and fluid-type detection as described above, singly or in any combination, where the separate photo-sensor may supplement the camera to provide more efficient high-drop-rate detection, backup functionality, or both. A separate illuminator, possibly operating at a separate wavelength, may be used in conjunction with a camera or a separate photo-sensor for redundant operation, improved fluid type detection, or other functionality.

The imaging method described here in all alternative forms may also be used in conjunction with the weight sensor previously described to provide an additional level of accuracy and redundancy. The imaging method provides sub-drop measurement but generally is not intended to provide highly precise measurement of absolute drop volume. Use of a weight sensor can provide long term volume correction in applications where absolute delivery volume is critical.

Advantages for the Imaging Method Described Herein:

The flow rate detection method described here provides several advantages over previous methods. It provides sub-drop resolution for more accurate measurement and control. It provides free-flow detection. It can differentiate between aqueous and lipid solutions as well as other opaque solutions. It requires relatively low power usage and processing requirements. Detection of pendant drops (rather than falling drops) requires lower reading frequency to ensure that no drops are missed. Detection of pendant drops reduces the possibility that a drop will be missed due to tilting of the drip chamber. Positioning of optics at the higher drop-forming position reduces optical interference due to droplets or misting, which typically occur lower on the drip chamber internal walls. It allows a variety of specific configurations for optimal performance for specific or specialized applications.

Another embodiment is similar to previous embodiments except that it employs a digital camera to monitor the level of the fluid pool at the bottom of the drip chamber. As shown in FIG. 14A, fluid pool 1403 is maintained in the lower part of drip chamber body 402. Lower camera 1406 is directed generally horizontally, adjacent to and directed toward fluid pool 1403 so that the meniscus formed at the top surface of the fluid pool is within the field of view of the camera. Digital images obtained from the camera are processed by connected electronic or computer components to obtain a metric indicating the level or depth of the fluid pool.

It will be evident to one skilled in the art that many variations can be made to the present invention while maintaining its novel features and functionality. In particular, the various motive means, sensor means, and geometric variations of the embodiments described above may be used in various combinations to create additional embodiments. Further, the components used in the present invention may be fabricated from a range of materials, including various polymers, metals, ceramics, and natural substances, and manufactured by various means, including molding, machining, printing, forming, and others, while maintaining its novel features and functionality. Further, the orientations of components used in the drawings and descriptions herein has been chosen only the purpose of clarity of explanation.

Although the foregoing disclosure has laid out various embodiments of an apparatus for controlling the flow rate of a fluid into a patient's body, it is to be understood that this disclosure is intended to also cover the related methods for controlling the flow rate of a fluid into a patient's body which may readily be inferred by those of ordinary skill in the art from the apparatus embodiments disclosed.

The knowledge possessed by someone of ordinary skill in the art at the time of this disclosure, including but not limited to the prior art disclosed with this application, is understood to be part and parcel of this disclosure and is implicitly incorporated by reference herein, even if in the interest of economy express statements about the specific knowledge understood to be possessed by someone of ordinary skill are omitted from this disclosure. While reference may be made in this disclosure to the invention comprising a combination of a plurality of elements, it is also understood that this invention is regarded to comprise combinations which omit or exclude one or more of such elements, even if this omission or exclusion of an element or elements is not expressly stated herein, unless it is expressly stated herein that an element is essential to applicant's combination and cannot be omitted. It is further understood that the related prior art may include elements from which this invention may be distinguished by negative claim limitations, even without any express statement of such negative limitations herein. It is to be understood, between the positive statements of applicant's invention expressly stated herein, and the prior art and knowledge of the prior art by those of ordinary skill which is incorporated herein even if not expressly reproduced here for reasons of economy, that any and all such negative claim limitations supported by the prior art are also considered to be within the scope of this disclosure and its associated claims, even absent any express statement herein about any particular negative claim limitations.

Finally, while only certain preferred features of the invention have been illustrated and described, many modifications, changes and substitutions will occur to those skilled in the art. It is, therefore, to be understood that the appended claims are intended to cover all such modifications and changes as fall within the true spirit of the invention.

### APPENDIX: LAMINAR FLOW IN CIRCULAR TUBES WITH MULTIPLE DIAMETERS

Laminar flow through a circular tube or pipe is described by:

$$Q = \frac{Δ P π {D}^{4}}{128 μL}$$

where Q is the volumetric flow rate, ΔP is the pressure difference between the two ends of the tube, D is the inside diameter of the tube μ is the dynamic viscosity of the fluid, and L is the length of the tube.

This can be rearranged as:

$$Q = \frac{Δ P}{{R}_{f}} where {R}_{f} = \frac{128 μL}{π {D}^{4}} ,$$

analogous to Ohm's law in electronics, with R_{f} being the fluid resistance of the tube. If two or more tubes, possibly have different lengths and diameters, are connected in series the total flow resistance is simply the addition of the individual flow resistance, giving:

$$Q = \frac{Δ P}{{R}_{f} ( {D}_{1} , {L}_{1} ) + {R}_{f} ( {D}_{2} , {L}_{2} ) + …}$$

where R_{f} is considered a function of diameter and length. This formula may be applied, for example to determine the minimum flow resistance for an intravenous delivery where no occlusion is applied and an IV set tube and IV needle are connected in series.

The true value for ΔP in a gravity-driven infuser is determined by the head height from the top of the fluid surface in the container to the entry point on the patient, less the internal pressure of the patient's vein. Peripheral venous pressure for a prone or seated person is normally less than 1000 Pa. The pressure is thus given by:

ΔP=ρgh−Pᵥ

where ρ is the density of the fluid, g is the acceleration of gravity, and his the height difference between the upper and lower fluid surfaces and Pᵥ is the internal venous pressure. For a 1 m head height the total pressure is therefore at least 8800 Pa. The internal pressure is not negligible but is a small fraction of the head height pressure under normal operating conditions.

I claim:

1. A medical infusion device for controlling the flow rate of a fluid into a patient's body, comprising:
a tube for carrying fluid from a proximal end to a distal end thereof under the action of a driving pressure, which tube is flexible or has a flexible segment at some point along its length;
a clamping element capable of preventing fluid flow by fully occluding a portion of said flexible tube or said flexible segment;
a movable pusher element for acting variably against said clamping element to variably reduce said occlusion of the clamped portion of said flexible tube or flexible segment and thereby provide a controlled rate of fluid flow;
two independently controllable electromechanically controlled actuator elements capable of moving variably over a prescribed range; and
a mechanical linkage among said actuator elements, said pusher element and said clamping element, configured such that the motion of said actuator elements is transmitted to said pusher element which in turn is transmitted to said clamping element for varying said occlusion and thereby varying said rate of fluid flow; wherein:
the motion of said pusher element is a function of the motions of said actuator elements; and
the force applied by the pusher element is a function of the force applied by said actuator elements.

2. The device of claim 1, said mechanical linkage comprising:
a coupling arm with two end points thereof;
each of said two independently controllable actuator elements situated adjacent and configured to act upon and move, a respective one of said coupling arm end points;
said pusher element situated adjacent and configured to act upon and move, an intermediate point of said coupling arm, wherein:
said motion of said pusher element is a function that is a weighted average of the movements of said actuator elements, as determined by the relative lengths of the two portions of said coupling arm between said intermediate point and said end points; and
force applied by said pusher element is a function that is a weighted sum of the force applied by said actuator elements, as determined by the relative lengths of the two portions of said coupling arm between said intermediate point and said end points.

3. The device of claim 2, each said actuator element comprising a motor for acting directly or through a gear reducer to rotate a cam, wherein:
said cam contacts a cam follower or fixed surface at one of said end points of said coupling arm to produce movement of said end point of said coupling arm.

4. The device of claim 1, each actuator element comprising a bellows or cylinder for extending under controlled pneumatic or hydraulic pressure, and retracting when no pressure is applied by the action of a return spring.

5. The device of claim 1, further comprising two independent electronic actuator controllers, each independently controlling one of said two independently controllable electromechanically controlled actuator elements; configured wherein:
a failure of one of said actuator controllers does not affect the operation of the other actuator controller and its associated actuator; and
each of said actuator controllers receives status signals from other elements of a flow control device, including the other actuator controller and a master controller, and can independently respond to signals indicating error or failure conditions occurring in said other elements.

6. The device of claim 1, wherein:
said actuator elements, said pusher element, and said mechanical linkage are configured such that:
both actuators must move a prescribed distance to cause movement of said pusher element sufficiently so as to enable fluid flow; and
either of said actuator elements is configured such that it can be moved to a particular position which will in turn move said pusher element into a disposition that fully occludes fluid flow, independent of the movement or position of the other of said actuator elements.

7. The device of claim 1, further comprising a drip chamber and a flow rate monitoring system, said flow rate monitoring system comprising:
at least one optical imager directed toward areas and fluid flow features within said drip chamber;
at least one illuminator for directing illumination toward features within said drip chamber to which said optical imagers are directed;
a user interface, computerized or electronic processing, and non-transient computerized storage capable of performing processing and analysis operations and extracting feature information from digital images obtained by said optical imagers; and
said computerized or electronic processing further capable of analyzing and obtaining metrics from said feature information.

8. The device of claim 7, said fluid flow features within said drip chamber selected from the fluid flow features group consisting of: fluid entering said drip chamber at a nozzle of said chamber during use; pendant fluid drops in area below said nozzle where pendant fluid drops form during use; and a fluid pool in lower section of said drip chamber formed during use.

9. The device of claim 7, said metrics for said drip chamber selected from the metric group consisting of: drop rate; fluid pool depth; fluid type; pendant drop volume; error conditions; label data; and tag data.

10. The device of claim 7, wherein growth of pendant drops is monitored as a means for measuring flow rate in sub-drop increments and further comprising:
said fluid flow features comprising pendant fluid drops in area below said nozzle where pendant fluid drops form during use;
said at least one optical imager and said at least one illuminator configured such that a distinct specular highlight appears in images obtained with said optical imager, and such that said specular highlight is displaced downward as each of said pendant drops increases in volume;
said computerized or electronic processing capable of converting said specular highlights in said images to a metric comprising pendant drop volume; and capable of thereby calculating flow rate in sub-drop increments.

11. A medical infusion device for delivering of fluids with controlled flow rate and volume into a patient's body, especially in environments where the presence of ferromagnetic material and the generation magnetic field related to such a device must be minimized, comprising:
a tube for carrying fluid from a proximal end to a distal end thereof under the action of a driving pressure, which is capable of being connected to a patient at said distal end;
said proximal end elevated above said distal end such that gravity applies a differential pressure across a length of said tube, causing fluid to flow through said tube from said proximal to said distal end;
omitting any magnetic motor pump for causing fluid to flow across said length of said tube;
omitting any non-magnetic motor pump for causing fluid to flow across said length of said tube;
a valve element at an intermediate point along said tube, capable of varying fluid flow by variably occluding a flow path through said tube, thereby variably controlling the rate of fluid flow;
a valve manipulating element capable of being variably moved relative to said valve element for causing said valve element to produce a variable occlusion of said tube;
at least one non-magnetic electrical motor for producing a mechanical output of variable movement and motive force;
a mechanical linkage between said motors and said valve manipulating element, for controllably moving said valve element by converting output motion of said at least one motor to motion of said valve manipulating element;
said mechanical linkage comprising a mechanical reducer for increasing the output motive force of said motors and decrease the velocity and displacement output of said motors to improve the resolution of movement of said valve element beyond the resolution achieved absent said mechanical reducer;
non-magnetic position sensing means, coupled to at least one of: said motors, said mechanical linkage, and said valve manipulating element, configured to provide feedback to a motor controller to allow controlled movement of said at least one non-magnetic electrical motor; and
for each of said non-magnetic electrical motors, two electrical transformers for increasing driving voltage, which transformers have a reduced size, reduced current capacity, and reduced output voltage, and which thereby generate reduced magnetic fields, in relation to transformers that would be required if a non-magnetic motor pump was not omitted; wherein:
said at least one non-magnetic electrical motor is configured to operate only when a change or correction of flow rate is required, thereby using less energy than would be used by a continuously-operating motor.

12. The device of claim 11, said at least one non-magnetic electrical motor selected from the non-magnetic motor group consisting of: piezoelectric motors; and ultrasonic motors.

13. The device of claim 11, further comprising a drip chamber and a flow rate monitoring system, said flow rate monitoring system comprising:
at least one optical imager directed toward areas and fluid flow features within said drip chamber;
at least one illuminator for directing illumination toward features within said drip chamber to which said optical imagers are directed;
a user interface, computerized or electronic processing, and non-transient computerized storage capable of performing processing and analysis operations and extracting feature information from digital images obtained by said optical imagers; and
said computerized or electronic processing further capable of analyzing and obtaining metrics from said feature information.

14. The device of claim 13, said fluid flow features within said drip chamber selected from the fluid flow features group consisting of: fluid entering said drip chamber at a nozzle of said chamber during use; pendant fluid drops in area below said nozzle where pendant fluid drops form during use; and a fluid pool in lower section of said drip chamber formed during use.

15. The device of claim 13, said metrics for said drip chamber selected from the metric group consisting of: drop rate; fluid pool depth; fluid type; pendant drop volume; error conditions; label data; and tag data.

16. The device of claim 13, wherein growth of pendant drops is monitored as a means for measuring flow rate in sub-drop increments and further comprising:
said fluid flow features comprising pendant fluid drops in area below said nozzle where pendant fluid drops form during use;
said at least one optical imager and said at least one illuminator configured such that a distinct specular highlight appears in images obtained with said optical imager, and such that said specular highlight is displaced downward as each of said pendant drops increases in volume;
said computerized or electronic processing capable of converting said specular highlights in said images to a metric comprising pendant drop volume; and capable of thereby calculating flow rate in sub-drop increments.

17. A separable portion device of a medical infusion device for controlling the flow rate of a fluid into a patient's body, said separable portion device comprising:
a flexible tube having fluid connection fittings of standard type at proximal and distal ends;
an clamp enclosure containing a movable clamping element at an intermediate point along the length of said tube;
an opening in said enclosure through which a portion of said clamp element protrudes or is otherwise accessible, such that said clamp element may be actuated by a component of said device to variably occlude and thereby to control the rate of flow of fluid through said flexible tube;
a drip chamber at an intermediate point along the tube length and proximate to said clamp enclosure; and
a plurality of mounting surfaces on said clamp enclosure and said drip chamber such that these elements can be affixed and aligned to said device.

18. The device of claim 17, said clamp enclosure and said drip chamber comprising a single assembly, wherein said single assembly is mounted to a fluid flow control device by a first movement to set said assembly in place in a holder on said flow device and a second movement to engage elements on said assembly to elements on said holder on said device such that said assembly is fixed in place on said device.

19. The device of claim 18, wherein said first movement is a linear translation of said assembly and said second movement is a rotation of said assembly about its vertical axis.

20. The device of claim 17, wherein:
said enclosure is prevented from being mounted on a flow control device if a state of said flow control device is such that flow would be initiated immediately upon mounting; and
wherein said clamp enclosure may always be dismounted independently of a state of said flow control device.
